(19) United States
(12) Patent Application Publication
(10) Pub. No.: US 2006/0281807 A1
(43) Pub. Date: Dec. 14, 2006

(54) Quassinoid compositions for the treatment of cancer and other proliferative diseases

(75) Inventors: James D. McChesney, Boulder, CO (US); Piotr Bartyzel, Longmont, CO (US); Jonathan E. Foster, Arvada, CO (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Tapestry Pharmaceuticals, Inc., Boulder, CO (US)

(21) Appl. No.: 11/151,625

(22) Filed: Jun. 13, 2005

(51) Int. Cl. A61K 31/366; C07D 493/14

(52) U.S. Cl. 514/453; 549/275

(57) ABSTRACT

The invention provides quassinoid compounds that are useful in treating cancer. The invention further provides a composition comprising a pharmaceutically suitable carrier and at least one compound of the invention, a method of killing a cancer cell, and a method of treating cancer in a mammal.

### FIELD OF THE INVENTION

This invention pertains to quassinoid compounds, compositions thereof, and a method of treating cancer with at least one quassinoid compound.

### BACKGROUND OF THE INVENTION

The botanical family Simaroubaceae includes numerous species distributed primarily in pantropical regions. These plant species have been the source of a large family of bitter terpenoid substances collectively termed quassinoids. The discovery of a wide spectrum of biological properties for the quassinoids including anti-leukemic, anti-viral, anti-amoebic, and anti-malarial activities have sparked an intense interest in the quassinoids as potential human therapeutic agents.

The majority of quassinoids, which are sometimes referred to as simaroubolides, are heavily oxygenated lactones that include a carbon skeleton called “type I.”

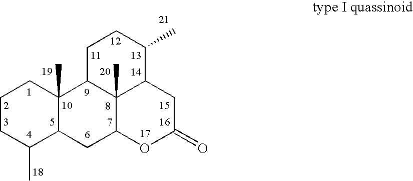

Quassinoids of type I are conventionally termed picrasane and can be further subdivided into three groups: group A, characterized by an oxide bridge between positions 11 and 20, group B, characterized by an oxide bridge between positions 13 and 20, and group C, which does not possess an oxide bridge (Cassady et al. (Eds.) Anticancer Agents Based on Natural Product Models, Academic Press, New York (1980)). Group A and B quassinoids have demonstrated antitumor activity, while Group C quassinoids are generally devoid of antitumor activity. Among the Group B quassinoids are compounds that can be considered derivatives of bruceolide, which features a hydroxyl group at C-15.

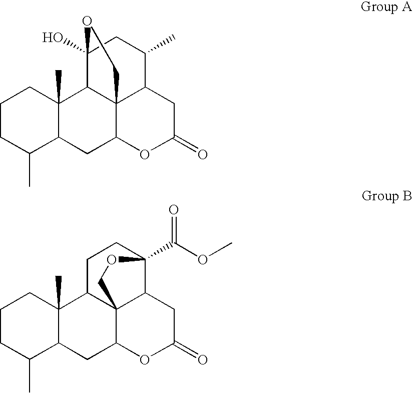

Of the Group B quassinoids, bruceantin was selected for clinical trials in humans in 1977 because of its significant cytotoxicity against several animal tumor systems. However, bruceantin did not progress beyond Phase II trials because of insufficient efficacy at the dose-limiting toxicity (DLT).

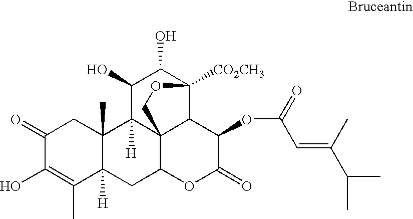

Recent research had demonstrated that quassinoids induce apoptosis (cell death) in lymphoma, myeloma, and leukemic cancer cells (see, for example, Kupchan et al., J. Med. Chem., 19(9): 1130-1133 (1976), Cassady et al. (Eds.) Anticancer Agents Based on Natural Product Models, Academic Press, New York (1980)). Possible mechanisms by which quassinoids exert their apoptotic effects include, for example, C-MYC downregulation, caspase activation, BID and PARP cleavage, and mitochondrial membrane depolarization (see, e.g., Cuendet et al., Clinical Cancer Research, 10, 1170-1179 (2004)).

Despite the efficacy demonstrated in vitro by members of the quassinoid family, there remains a need for agents having improved potency and selectivity for tumor cells. In addition, cancer cells often acquire resistance to chemotherapeutic drugs, primarily through activation of the multi-drug resistance mechanism within the cancer cells, creating a need for the identification of new agents to circumvent the resistance. Furthermore, natural products as isolated often require optimization of their structures to become useful clinical agents suitable for administration to a wide spectrum of patients.

Accordingly, there remains a need for more effective agents and methods for treating cancer, particularly in humans. The invention provides such compounds and methods. These and other advantages of the invention, as well as additional inventive features, will be apparent from the description of the invention provided herein.

### BRIEF SUMMARY OF THE INVENTION

The invention provides compounds of the formula (I)

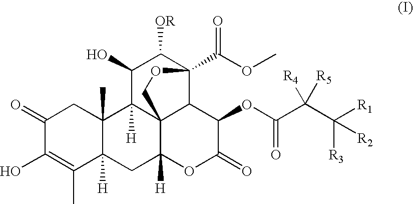

wherein R is selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl,

wherein R₁, R₂, and R₃ are independently selected from the group consisting of hydrogen, halo, alkyl, alkenyl, alkynyl, cycloalkyl, cycloalkenyl, aryl, arylalkyl, carboxyl, carbonyl, hydroxyalkyl, alkoxyalkyl, —OR₆, and —NR₉R₁₀, or wherein R₁ and R₂ taken together form C₃-C₁₀ alkylene, C₃-C₁₀ alkenylene, C₃-C₁₀ heterocycloalkyl, or C₃-C₁₀ heteroaryl.

wherein R₄ and R₅ are independently selected from the group consisting of hydrogen, alkyl, alkenyl, alkynyl, hydroxyalkyl, alkoxyalkyl, cycloalkyl, cycloalkenyl, aryl, and arylalkyl, or wherein R₄ and R₅ taken together form C₃-C₁₀ alkylene or alkenylene, or wherein R₄ and R₅ taken together form (C═O),

wherein R₆ is selected from the group consisting of hydrogen, alkyl, alkenyl, alkynyl, hydroxyalkyl, alkoxyalkyl, cycloalkyl, cycloalkenyl, aryl, arylalkyl, and —C(O)R₇,

wherein R₇ is selected from the group consisting of hydrogen, alkyl, alkenyl, alkynyl, hydroxyalkyl, alkoxyalkyl, cycloalkyl, cycloalkenyl, aryl, arylalkyl, —OR₈, and —NR₉R₁₀,

wherein R₈ is selected from the group consisting of alkyl, alkenyl, alkynyl, cycloalkyl, cycloalkenyl, aryl, and arylalkyl,

wherein R₉ and R₁₀ are independently selected from the group consisting of hydrogen, alkyl, alkenyl, alkynyl, hydroxyalkyl, alkoxyalkyl, cycloalkyl, cycloalkenyl, aryl, aralkyl, alkylcarbonyl, and arylcarbonyl, or R₉ and R₁₀ can be taken together to form a 5-7 membered heterocyclic ring with the nitrogen to which they are bonded,

with the provisos (i) R₁, R₂, R₃, R₄ and R₅ are not all hydrogen; (ii) when R₄ and R₅ are hydrogen and R₃ is hydroxyl, then R₁ and R₂ are not both methyl; (iii) if R₄ and R₅ are hydrogen and R₁ is methyl, then R₂ is not hydrogen and R₃ is not trifluoromethyl, (iv) when R₄ and R₅ are hydrogen and R₁ is isopropyl, then R₂ is not hydrogen and R₃ is not methyl, and (v) when R₃, R₄ and R₅ are hydrogen, then R₁ and R₂ are not both methyl.

The invention further provides compounds of the formula (II)

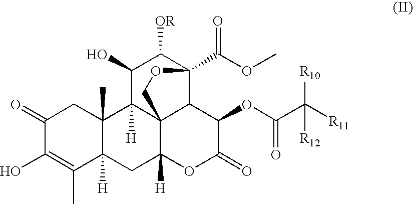

wherein R is selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl,

wherein R₁₀, R₁₁, and R₁₂ are independently selected from the group consisting of hydrogen, halo, alkyl, alkenyl, alkynyl, cycloalkyl, cycloalkenyl, aryl, arylalkyl, carboxyl, carbonyl, hydroxyalkyl, alkoxyalkyl, and —OR₁₃, or wherein R₁₀ and R₁₁ taken together form C₃-C₁₀ alkylene, C₃-C₁₀ alkenylene, or C₃-C₁₀ heterocycloalkyl,

wherein R₁₃ is selected from the group consisting of hydrogen, alkyl, alkenyl, alkynyl, hydroxyalkyl, alkoxyalkyl, cycloalkyl, cycloalkenyl, aryl, arylalkyl, and —C(O)R₁₄,

wherein R₁₄ is selected from the group consisting of alkyl, alkenyl, alkynyl, hydroxyalkyl, alkoxyalkyl, cycloalkyl, cycloalkenyl, aryl, arylalkyl, —OR₁₅, and —NR₁₆R₁₇,

wherein R₁₅ is selected from the group consisting of alkyl, alkenyl, alkynyl, cycloalkyl, cycloalkenyl, aryl, and arylalkyl,

wherein R₁₆ and R₁₇ are independently selected from the group consisting of hydrogen, alkyl, alkenyl, alkynyl, hydroxyalkyl, alkoxyalkyl, cycloalkyl, cycloalkenyl, aryl, and arylalkyl, or R₁₆ and R₁₇ can be taken together to form a 5-7 membered heterocyclic ring with the nitrogen to which they are bonded,

with the proviso that R, R₁₀, R₁₁, and R₁₂ are not all hydrogen.

The invention further provides a compound of the formula (III)

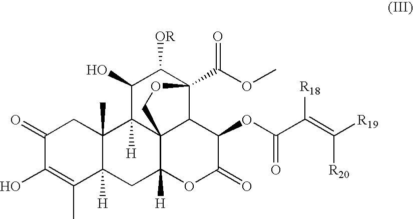

wherein R is selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl,

wherein R₁₈, R₁₉, and R₂₀ are independently selected from the group consisting of hydrogen, halo, alkyl, alkenyl, alkynyl, cycloalkyl, cycloalkenyl, aryl, arylalkyl, carboxyl, carbonyl, hydroxyalkyl, alkoxyalkyl, and —OR₂₁, or wherein R₁₉ and R₂₀ taken together form C₃-C₁₀ alkylene, C₃-C₁₀ alkenylene, or C₃-C₁₀ heterocycloalkyl,

wherein R₂₁ is selected from the group consisting of hydrogen, alkyl, alkenyl, alkynyl, hydroxyalkyl, alkoxyalkyl, cycloalkyl, cycloalkenyl, aryl, arylalkyl, and —C(O)R₂₂,

wherein R₂₂ is selected from the group consisting of alkyl, alkenyl, alkynyl, hydroxyalkyl, alkoxyalkyl, cycloalkyl, cycloalkenyl, aryl, arylalkyl, —OR₂₃, and —NR₂₄R₂₅,

wherein R₂₃ is selected from the group consisting of alkyl, alkenyl, alkynyl, cycloalkyl, cycloalkenyl, aryl, and arylalkyl,

wherein R₂₄ and R₂₅ are independently selected from the group consisting of hydrogen, alkyl, alkenyl, alkynyl, hydroxyalkyl, alkoxyalkyl, cycloalkyl, cycloalkenyl, aryl, and arylalkyl, or R₂₄ and R₂₅ can be taken together to form a 5-7 membered heterocyclic ring with the nitrogen to which they are bonded,

with the proviso when R₁₈ is hydrogen and R₁₉ is methyl, then R₂₀ is not methyl, isopropyl, —C(CH₃)₂OH, or —C(CH₃)₂OAc.

The invention further provides a compound of the formula (IV) 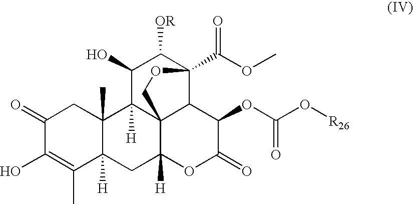
wherein R and R₂₆ are independently selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl.

The invention also provides a composition comprising a pharmaceutically suitable carrier and at least one compound of the invention.

The invention further provides a method of treating cancer comprising administering an effective amount of at least one compound of formula (I), (II), (III), or (IV) or a composition thereof to a mammal in need thereof, whereupon the cancer is treated.

### BRIEF DESCRIPTION OF THE DRAWINGS

FIG. 1 illustrates the synthesis of 15-O-(3′-hydroxy-3′-isopropyl-4′-methylpentanoyl)bruceolide starting from a mixture of bruceolides isolated from a natural source.

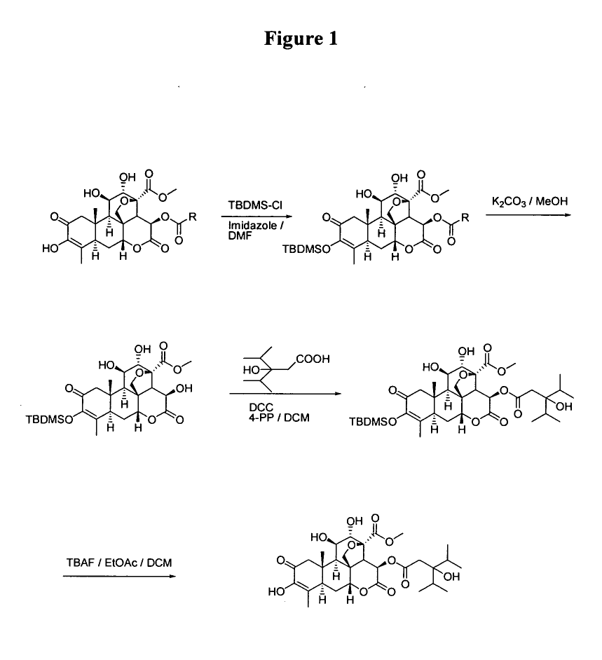

FIG. 2 is a graph which illustrates the cytotoxicity of 15-O-(3′-hydroxy-3′-isopropyl-4′-methylpentanoyl)bruceolide as a function of concentration. ▴ is an initial measurement; and ▪ is measured 10 weeks after the initial measurement.

FIG. 3 is a graph which illustrates the cytotoxicity of 15-O-(3-isopropyl-4-methyl-2-pentenoyl)-bruceolide as a function of concentration. ▴ is an initial measurement; and ▪ is measured 10 weeks after the initial measurement.

FIG. 4 is a graph which illustrates the clonogenic inhibition of 15-O-(3′-hydroxy-3′-isopropyl-4′-methylpentanoyl)bruceolide as a function of concentration. ▪ is measured at 2 hr; ♦ is measured at 24 hr; ▴ is measured at 168 hr; and ● is measured at 168 hr (repeat).

FIG. 5 is a graph which illustrates the clonogenic inhibition of 15-O-(3-isopropyl-4-methyl-2-pentenoyl)-bruceolide as a function of concentration. ▪ is measured at 2 hr; ♦ is measured at 24 hr; ▴ is measured at 168 hr; and ● is measured at 168 hr (repeat).

FIG. 6 is a graph which illustrates identification of the maximum tolerated dose of 15-O-(3′-hydroxy-3′-isopropyl-4′-methylpentanoyl)bruceolide. ♦ is at a dose of 3.0 mg/mouse; and ▪ is at a dose of 2.25 mg/mouse.

FIG. 7 is a graph which illustrates the concentration of 15-O-(3′-hydroxy-3′-isopropyl-4′-methylpentanoyl)bruceolide over time in vivo. A dose of 2.25 mg/mouse was administered intravenously, and at the twenty-fourth time point, the animals were dead. ♦ was the concentration in the plasma; and ▪ was the concentration in the tumor. The limit of sensitivity was 0.05 μg/mL.

FIG. 8 is a graph which illustrates the concentration of 15-O-(3-isopropyl-4-methyl-2-pentenoyl)-bruceolide over time in vivo. A dose of 0.075 mg/mouse was administered intravenously. Time points 1 to 24 hours were below the detection limit of 0.05 μg/mL. The level of 15-O-(3-isopropyl-4-methyl-2-pentenoyl)-bruceolide in the tumor was below the detection limit at all time points.

FIG. 9 is a graph which illustrates tumor volume over time in response to administration of 15-O-(3′-hydroxy-3′-isopropyl-4′-methylpentanoyl)bruceolide. ♦ is the control; ▪ is at a dose of 0.8 mg/mouse; and ▴ is at a dose of 0.4 mg/mouse.

### DETAILED DESCRIPTION OF THE INVENTION

The invention provides a compound of the formula (I)

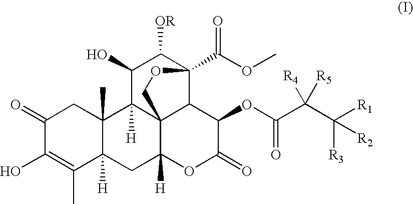

wherein R is selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl,

wherein R₁, R₂, and R₃ are independently selected from the group consisting of hydrogen, halo, alkyl, alkenyl, alkynyl, cycloalkyl, cycloalkenyl, aryl, arylalkyl, carboxyl, carbonyl, hydroxyalkyl, alkoxyalkyl, —OR₆, and —NR₉R₁₀, or wherein R₁ and R₂ taken together form C₃-C₁₀ alkylene, C₃-C₁₀ alkenylene, C₃-C₁₀ heterocycloalkyl, or C₃-C₁₀ heteroaryl,

wherein R₄ and R₅ are independently selected from the group consisting of hydrogen, alkyl, alkenyl, alkynyl, hydroxyalkyl, alkoxyalkyl, cycloalkyl, cycloalkenyl, aryl, and arylalkyl, or wherein R₄ and R₅ taken together form C₃-C₁₀ alkylene or alkenylene, or wherein R₄ and R₅ taken together form (C═O),

wherein R₆ is selected from the group consisting of hydrogen, alkyl,alkenyl, alkynyl, hydroxyalkyl, alkoxyalkyl, cycloalkyl, cycloalkenyl, aryl, arylalkyl, and —C(O)R₇,

wherein R₇ is selected from the group consisting of hydrogen, alkyl, alkenyl, alkynyl, hydroxyalkyl, alkoxyalkyl, cycloalkyl, cycloalkenyl, aryl, arylalkyl, —OR₈, and —NR₉R₁₀,

wherein R₈ is selected from the group consisting of alkyl, alkenyl, alkynyl, cycloalkyl, cycloalkenyl, aryl, and arylalkyl,

wherein R₉ and R₁₀ are independently selected from the group consisting of hydrogen, alkyl, alkenyl, alkynyl, hydroxyalkyl, alkoxyalkyl, cycloalkyl, cycloalkenyl, aryl, aralkyl, alkylcarbonyl, and arylcarbonyl, or R₉ and R₁₀ can be taken together to form a 5-7 membered heterocyclic ring with the nitrogen to which they are bonded,

with the provisos (i) R₁, R₂, R₃, R₄ and R₅ are not all hydrogen; (ii) when R₄ and R₅ are hydrogen and R₃ is hydroxyl, then R₁ and R₂ are not both methyl; (iii) if R₄ and R₅ are hydrogen and R₁ is methyl, then R₂ is not hydrogen and R₃ is not trifluoromethyl, (iv) when R₄ and R₅ are hydrogen and R₁ is isopropyl, then R₂ is not hydrogen and R₃ is not methyl and (v) when R₃, R₄ and R₅ are hydrogen, then R₁ and R₂ are not both methyl. Examples of compounds excluded from the invention by way of the proviso include certain compounds isolated from natural sources (e.g., Brucea antidysenterica), such as, for example, bruceantin, bruceantinol, brucein A, and dihydrobruceantin.

In a preferred embodiment, R₁ and R₂ are independently selected from the group consisting of hydrogen, alkyl, alkenyl, alkynyl, cycloalkyl, cyloalkenyl, aryl, arylalkyl, carboxyl, carbonyl, hydroxyalkyl, and alkoxyalkyl, and R₃ is —OR_{6.}

In a preferred compound of formula (I), R is hydrogen, R₄ and R₅ are hydrogen, R₁ and R₂ are both ethyl or isopropyl, and R₃ is hydroxyl or wherein R₁, R₂, and R₃ are each methyl.

An especially preferred compound of formula (I) includes R is hydrogen, R₄ and R₅ are hydrogen, R₁ and R₂ are isopropyl, and R₃ is hydroxyl. Such an exemplary compound has the structure:

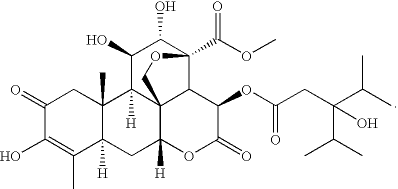

In other preferred compounds of formula (I), R₁ is hydroxyl, R₃ is hydrogen or methyl, and R₂ is carboxyl or lower alkyl carboxylic acid ester.

In still other preferred compounds of formula (I), R₃ can be —(CH₂)ₙOR₆, in which n is an integer from 1-10 (e.g., 1, 2, 3, 4, 5, 6, 7, 8, 9, or 10) and wherein R₁, R₂, R₄, and R₅ are as previously defined. Preferably, n is an integer from 1-5 (e.g., 1, 2, 3, 4, or 5). Preferred compounds of formula (I), in which R₃ is —(CH₂)ₙOR₆ include compounds in which R₃ is hydroxymethyl, R₄ and R₅ are hydrogen, and R₁ and R₂ are both ethyl, isopropyl, or methyl.

Alternatively, R₄ and R₅ can be taken together to form ring structures (e.g., C₃₋₁₁ cycloalkyl) with the carbon to which they are bonded. For example, R₄ and R₅ can be taken together to form a four-membered ring, R₁ and R₂ are hydrogen, and R₃ is hydroxyl.

Also preferably, compounds of formula (I) include those in which R is hydrogen, alkyl, C(O)CH═C(CH₃)CH(CH₃)₂, C(O)OCH₂CH═CH₂, or 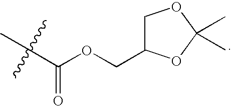
More preferably, R is hydrogen.

The invention further provides a compound of the formula (II)

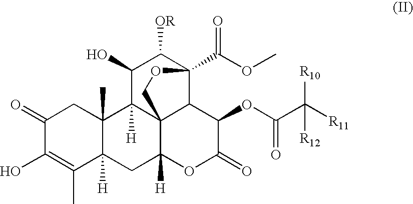

wherein R is selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl,

wherein R₁₀, R₁₁, and R₁₂ are independently selected from the group consisting of hydrogen, halo, alkyl, alkenyl, alkynyl, cycloalkyl, cycloalkenyl, aryl, arylalkyl, carboxyl, carbonyl, hydroxyalkyl, alkoxyalkyl, and —OR₁₃, or wherein R₁₀ and R₁₁ taken together form C₃-C₁₀ alkylene, C₃-C₁₀ alkenylene, or C₃-C₁₀ heterocycloalkyl,

wherein R₁₃ is selected from the group consisting of hydrogen, alkyl, alkenyl, alkynyl, hydroxyalkyl, alkoxyalkyl, cycloalkyl, cycloalkenyl, aryl, arylalkyl, and —C(O)R₁₄,

wherein R₁₄ is selected from the group consisting of alkyl, alkenyl, alkynyl, hydroxyalkyl, alkoxyalkyl, cycloalkyl, cycloalkenyl, aryl, arylalkyl, —OR₁₅, and —NR₁₆R₁₇,

wherein R₁₅ is selected from the group consisting of alkyl, alkenyl, alkynyl, cycloalkyl, cycloalkenyl, aryl, and arylalkyl,

wherein R₁₆ and R₁₇ are independently selected from the group consisting of hydrogen, alkyl, alkenyl, alkynyl, hydroxyalkyl, alkoxyalkyl, cycloalkyl, cycloalkenyl, aryl, and arylalkyl, or R₁₆ and R₁₇ can be taken together to form a 5-7 membered heterocyclic ring with the nitrogen to which they are bonded,

with the proviso that R, R₁₀, R₁₁, and R₁₂ are not all hydrogen. Examples of compounds excluded from the invention by way of the proviso include certain compounds isolated from natural sources (e.g., Brucea antidysenterica), such as, for example, bruceantarin and bruceine B.

In one embodiment of the invention, R₁₀ and R₁₁ are independently selected from the group consisting of alkyl, alkenyl, alkynyl, cycloalkyl, cycloalkenyl, aryl, arylalkyl, carboxyl, carbonyl, hydroxyalkyl, and alkoxyalkyl, and R₁₂ is —OR₁₃. In another embodiment, R₁₂ is —(CH₂)ₙOR₁₃, in which n is an integer of 1-10 (e.g., 1, 2, 3, 4, 5, 6, 7, 8, 9, or 10) and preferably, n is an integer from 1-5 (e.g., 1, 2, 3, 4, or 5).

Preferred compounds of formula (II) include those in which R is hydrogen, R₁₀, R₁₁, and R₁₂ are each methyl; R₁₀ and R₁₁ are methyl and R₁₂ is hydroxyl; and wherein R₁₀ and R₁₁ are isopropyl and R₁₂ is hydroxyl.

In other preferred compounds of formula (II), R is hydrogen, R₁₀ and R₁₁ are both methyl or isopropyl and R₁₂ is hydroxymethyl.

Also preferably, compounds of formula (II) include those in which R is hydrogen, alkyl, C(O)CH═C(CH₃)CH(CH₃)₂, C(O)OCH₂CH═CH₂, or 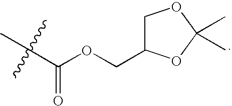
More preferably, R is hydrogen.

It will be appreciated that in some instances when R₁₀ and R₁₁ are taken together to form C₃-C₁₀ alkenylene (e.g., 1-cyclopentenyl or 1-cyclohexenyl), that R₁₂ will not be present in the compound if the carbon to which R₁₀ and R₁₁ are attached has no open valency for R₁₂.

The invention further provides a compound of the formula (III)

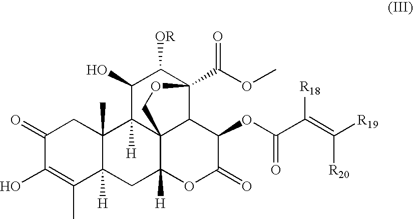

wherein R is selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl,

wherein R₁₈, R₁₉, and R₂₀ are independently selected from the group consisting of hydrogen, halo, alkyl, alkenyl, alkynyl, cycloalkyl, cycloalkenyl, aryl, arylalkyl, carboxyl, carbonyl, hydroxyalkyl, alkoxyalkyl, and —OR₂₁, or wherein R₁₉ and R₂₀ taken together form C₃-C₁₀ alkylene, C₃-C₁₀ alkenylene, or C₃-C₁₀ heterocycloalkyl,

wherein R₂₁ is selected from the group consisting of hydrogen, alkyl, alkenyl, alkynyl, hydroxyalkyl, alkoxyalkyl, cycloalkyl, cycloalkenyl, aryl, arylalkyl, and —C(O)R₂₂,

wherein R₂₂ is selected from the group consisting of alkyl, alkenyl, alkynyl, hydroxyalkyl, alkoxyalkyl, cycloalkyl, cycloalkenyl, aryl, arylalkyl, —OR₂₃, and —NR₂₄R₂₅,

wherein R₂₃ is selected from the group consisting of alkyl, alkenyl, alkynyl, cycloalkyl, cycloalkenyl, aryl, and arylalkyl,

wherein R₂₄ and R₂₅ are independently selected from the group consisting of hydrogen, alkyl, alkenyl, alkynyl, hydroxyalkyl, alkoxyalkyl, cycloalkyl, cycloalkenyl, aryl, and arylalkyl, or R₂₄ and R₂₅ can be taken together to form a 5-7 membered heterocyclic ring with the nitrogen to which they are bonded,

with the proviso when R and R₁₈ are hydrogen and R₁₉ is methyl, then R₂₀ is not methyl, isopropyl, —C(CH₃)₂OH, or —C(CH₃)₂OAc. Examples of compounds excluded from the invention by way of the proviso include certain compounds isolated from natural sources (e.g., Brucea antidysenterica), such as, for example, bruceantin, brucein C, bruceantinol, and brusatol.

Preferred compounds of formula (III) include those in which (i) R is hydrogen, R₁₈ is hydrogen and R₁₉ and R₂₀ are ethyl or isopropyl, (ii) R is hydrogen, R₁₈ and R₁₉ are hydrogen, and R₂₀ is phenyl, and (iii) R is hydrogen, R₁₈ is hydrogen and R₁₉ and R₂₀ taken together are cyclopentyl or cyclohexyl.

Also preferably, compounds of formula (III) include those in which R is hydrogen, alkyl, C(O)CH═C(CH₃)CH(CH₃)₂, C(O)OCH₂CH═CH₂, or 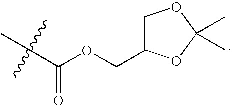
More preferably, R is hydrogen.

An especially preferred compound of formula (III) includes R is hydrogen, R₁₈ is hydrogen and R₁₉ and R₂₀ are isopropyl. Such an exemplary compound has the structure:

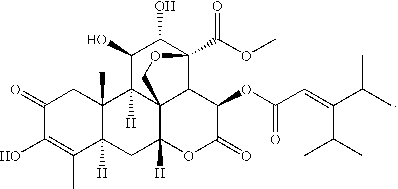

The invention further provides a compound of the formula (IV) 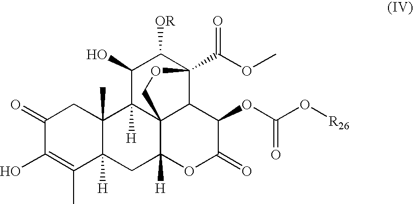
wherein R and R₂₆ are independently selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl.

Preferred compounds of formula (IV) include those in which R₂₆ is CH₂CH═CH₂ or 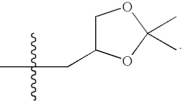
Also preferably, compounds of formula (IV) include those in which R is hydrogen, alkyl, C(O)CH═C(CH₃)CH(CH₃)₂, C(O)OCH₂CH═CH₂, or 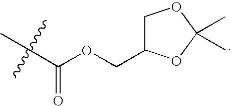
More preferably, R is hydrogen.

Any one or more of R₁₋₂₆ of formulae (I)-(IV) can be substituted. Generally each of R₁₋₂₆ can have 1 to 8 substituents (e.g., 1 to 6, 1 to 4, 1 to 3, 1 to 2 substituents) that are independently selected from the group consisting of C₁₋₁₂ alkyl, C₆₋₃₀ aryl, C₁₋₁₂ alkoxy, C₁₋₁₂ aryloxy, acyloxy, benzyl, benzyloxy, carboxyl, formyl, carboxy-C₁₋₁₂ alkyl, carboxy-C₁₋₁₂ alkylamido, carboxy-C₁₋₁₂ dialkylamido, carboxamido, C₁₋₁₂ alkylcarbonyl, phenylcarbonyl, benzylcarbonyl, C₆₋₃₀ arylamino, C₆₋₃₀ diarylamino, C₁₋₁₂ alkylthio, C₅₋₃₀ heteroaryl, such as pyranyl, pyrrolyl, furanyl, 2′,5′-dimethyl-3′-furanyl, thiophenyl, thiazolyl, pyrazolyl, pyridinyl, or pyrimidinyl, C₁₋₁₂ trialkylsilyl, pyrrolidinyl, dioxanyl, tetrahydrofuranyl, tetrahydropyranyl, piperdinyl, morpholinyl, nitro, sulfonyl, halo, cyano, hydroxy, thio, C₃₋₈ cycloalkyl, amino, C₁₋₁₂ alkylamino, and C₁₋₁₂ dialkylamino.

It will be appreciated that compounds of formula (I), (II), (III), or (IV) can exist in the form of a salt, such as a carboxylic acid salt (e.g., a Group I salt, a Group II salt, a metal salt, an ammonium salt, or the like) or an amine salt (e.g., an acid addition salt). For example, malonates include malonic acid, as well as mono- and di-salts thereof, and amines include the free base, as well as salts thereof. Furthermore, carboxylic acids including basic functional groups can exist in the form of an acid salt of the basic functional group. For example, glycines include glycine, as well as monoacid salts thereof. Furthermore, some compounds can function both as an acid and as a chelating agent (e.g., certain amino acids and the like).

Suitable acid addition salts can be prepared from pharmaceutically acceptable non-toxic acids including inorganic and organic acids. Such acids include acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic, and the like.

Referring now to terminology used generically herein, the term “alkyl” means a straight-chain or branched alkyl substituent containing from, for example, about 1 to about 12 carbon atoms, preferably from about 1 to about 8 carbon atoms, more preferably from about 1 to about 6 carbon atoms. Examples of such substituents include methyl, ethyl, propyl, isopropyl, n-butyl, sec-butyl, isobutyl, tert-butyl, pentyl, isoamyl, hexyl, octyl, dodecanyl, and the like. The term “hydroxyalkyl” means an alkyl group as described herein that is substituted with one or more hydroxyl (—OH) groups.

The term “alkenyl,” as used herein, means a linear alkenyl substituent containing at least one carbon-carbon double bond and from, for example, about 2 to about 12 carbon atoms (branched alkenyls are about 3 to about 12 carbons atoms), preferably from about 2 to about 8 carbon atoms (branched alkenyls are preferably from about 3 to about 8 carbon atoms), more preferably from about 3 to about 6 carbon atoms. Examples of such substituents include propenyl, isopropenyl, n-butenyl, sec-butenyl, isobutenyl, tert-butenyl, pentenyl, isopentenyl, hexenyl, octenyl, dodecenyl, and the like.

The term “alkynyl,” as used herein, means a linear alkynyl substituent containing at least one carbon-carbon triple bond and from, for example, about 2 to about 12 carbon atoms (branched alkynyls are about 3 to about 12 carbons atoms), preferably from about 2 to about 8 carbon atoms (branched alkynyls are preferably from about 3 to about 8 carbon atoms), more preferably from about 3 to about 6 carbon atoms. Examples of such substituents include propynyl, isopropynyl, n-butynyl, sec-butynyl, isobutynyl, tert-butynyl, pentynyl, isopentynyl, hexynyl, octynyl, dodecynyl, and the like.

The term “cycloalkyl,” as used herein, means a cyclic alkyl substituent containing from, for example, about 3 to about 30 carbon atoms, preferably from about 4 to about 14 carbon atoms, more preferably from about 4 to about 10 carbon atoms, and most preferably from about 5 to about 7 carbon atoms. Examples of such substituents include cyclopropyl, cyclobutyl, cyclopentyl, cyclohexyl, and the like. The term “cycloalkenyl,” as used herein, means the same as the term “cycloalkyl,” however one or more double bonds are present. Examples of such substituents include cyclopentenyl and cyclohexenyl.

The term “heterocycloalkyl,” as used herein, means a cyclic alkyl substituent containing from, for example, about 3 to about 30 carbon atoms, preferably from about 4 to about 14 carbon atoms, more preferably from about 4 to about 10 carbon atoms, and most preferably from about 5 to about 7 carbon atoms and from 1 to 3 heteroatoms (e.g., N, O, or S). Examples of such substituents include morpholino, epoxy, aziridinyl, pyrrolidinyl, piperidinyl, and piperazinyl.

The term “halo” or “halogen,” as used herein, means a substituent selected from Group VIIA, such as, for example, fluorine, bromine, chlorine, and iodine.

The term “aryl” refers to an unsubstituted or substituted aromatic carbocyclic substituent, as commonly understood in the art, and includes monocyclic and polycyclic aromatics such as, for example, phenyl, biphenyl, toluenyl, anisolyl, naphthyl, anthracenyl and the like. An aryl substituent generally contains from, for example, about 3 to about 30 carbon atoms, preferably from about 6 to about 18 carbon atoms, more preferably from about 6 to about 14 carbon atoms and most preferably from about 6 to about 10 carbon atoms. It is understood that the term aryl applies to cyclic substituents that are planar and comprise 4n+2 π electrons, according to Hückel's Rule. The term “arylalkyl” means an alkyl group as described herein that is substituted with one or more aryl groups as described herein.

The term “heteroaryl,” as used herein, means an aryl substituent containing from, for example, about 3 to about 30 atoms, preferably from about 6 to about 18 atoms, more preferably from about 6 to about 14 atoms, and most preferably from about 6 to about 10 atoms and from 1 to 3 heteroatoms (e.g., N, O, or S). Examples of such substituents include pyrrolyl, imidazolyl, pyrazolyl, furanyl, oxazolyl, isooxazolyl, thiofuranyl, thiazolyl, isothiazolyl, indolyl, isoindolyl, benzofuranyl, quinolinyl, pyridinyl, pyridazinyl, pyrazinyl, triazolyl, and benzotriazolyl.

The term “alkoxy” embraces linear or branched alkyl groups that are attached to divalent oxygen. The alkyl group is the same as described herein. Examples of such substituents include methoxy, ethoxy, t-butoxy, and the like. The term “alkoxyalkyl” refers to an alkyl group that is substituted with one or more alkoxy groups. The alkyl and alkoxy groups are as described herein.

The term “carboxyl” refers to the group —C(O)OH. The term “alkylcarboxy” refers to the group —C(O)R, wherein R is an alkyl group as described herein. Examples of such substituents include —C(O)CH₃ and —C(O)CH₂CH₃. Similarly, the term “arylcarboxy” refers to the group —C(O)R′, wherein R′ is an aryl group as described herein. An example of such substituents include —C(O)Ph.

The invention further provides a composition comprising a pharmaceutically suitable carrier and at least one compound of formula (I), (II), (III), or (IV). Preferably, the composition comprises a therapeutically effective amount of a compound of the invention. Any suitable carrier can be used within the context of the invention, many of which are well known in the art. The choice of carrier will be determined, in part, by the particular method used to administer the pharmaceutical compositions and by the particular site to which the pharmaceutical compositions are to be administered.

In an exemplary embodiment, the composition comprises the compound of the formula 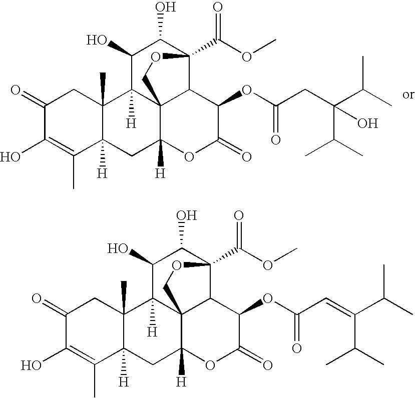
and a pharmaceutically suitable carrier.

Suitable formulations include aqueous and non-aqueous solutions, isotonic sterile solutions, which can contain anti-oxidants, buffers, bacteriostats, and solutes that render the formulation isotonic with the blood or other bodily fluid of the intended recipient, and aqueous and non-aqueous sterile suspensions that can include suspending agents, solubilizers, thickening agents, stabilizers, and preservatives. In one embodiment, the pharmaceutically acceptable carrier is a liquid that contains a buffer and a salt. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. In one embodiment, the pharmaceutically acceptable carrier is a buffered saline solution. Raymond C. Rowe, Paul J. Sheskey, and Paul J. Weller, Eds., Handbook of Pharmaceutical Excipients, 4^{th} Ed., Pharmaceutical Press: London, 2003, is incorporated by reference herein and provides examples of suitable carriers.

Further carriers include sustained-release preparations, such as semipermeable matrices of solid hydrophobic polymers containing the active agent, which matrices are in the form of shaped articles (e.g., films, liposomes, or microparticles). The pharmaceutical composition comprising the compound of formula (I), (II), (III), or (IV) can include carriers, thickeners, diluents, buffers, preservatives, surface active agents and the like. The pharmaceutical compositions can also include one or more additional active ingredients, such as other anticancer agents or chemotherapeutics, antimicrobial agents, anti-inflammatory agents, anesthetics, and the like.

The pharmaceutical composition comprising the compound of formula (I), (II), (III), or (IV) can be formulated for any suitable route of administration, depending on whether local or systemic treatment is desired, and on the area to be treated. Desirably, the pharmaceutical composition is formulated for parenteral administration, such as intravenous, intraperitoneal, intramuscular, or intratumoral injection. Injectables can be prepared in conventional forms, either as liquid solutions or suspensions, solid forms suitable for suspension in liquid prior to injection, or as emulsions. Additionally, parental administration can involve the preparation of a slow-release or sustained-release system, such that a constant dosage is maintained. Preparations for parenteral administration include sterile aqueous or non-aqueous solutions, suspensions, and emulsions. Examples of non-aqueous solvents are propylene glycol, polyethylene glycol, vegetable oils, such as olive oil, and injectable organic esters, such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives also can be present such as, for example, antimicrobials, anti-oxidants, chelating agents, and inert gases and the like.

The invention provides a method of killing a cancer cell, which method comprises contacting a cancer cell with at least one compound of formula (I), (II), (III), or (IV). Preferably, the cancer cell is contacted with a composition comprising a pharmaceutically acceptable carrier and a composition of formula (I), (II), (III), or (IV). The cancer cell can be contacted in vitro or in vivo. When the cancer cell is contacted with the inventive compound, the compound preferably contacts a sample that contains one or more cancer cells. The sample can be any suitable sample in which at least one cancer cell can be found. Examples of suitable samples include, tissue, blood, serum, or urine. In embodiments where the sample is a tissue, the tissue can be isolated from any suitable organ in a mammal (e.g., a human), including the heart, brain, breast, lungs, liver, kidney, reproductive organs (e.g., uterus), digestive organs (e.g., stomach, intestines), and the like.

In another embodiment, the cancer cell can be contacted with the inventive compound in vivo. In this regard, the invention provides a method of treating cancer in a mammal, which method comprises a dose of a composition comprising a pharmaceutically acceptable carrier and an effective amount of at least one compound of formula (I), (II), (III), or (IV) to a mammal, whereupon the cancer is treated. The inventive method can be used to treat any cancer in any suitable mammal (e.g., mouse, rat, monkey, or human). In one embodiment, the inventive method is used to treat cancer in a human.

The at least one compound of formula (I), (II), (III), or (IV) or a composition thereof preferably contacts a tumor associated with the cancer, resulting in the destruction of tumor cells within the tumor. The tumor can be a solid tumor or a tumor associated with soft tissue (i.e., soft tissue sarcoma), in a mammal. The term “tumor” refers to both tumor cells and associated stromal cells. The tumor can be associated with cancers of (i.e., located in) the oral cavity and pharynx, the digestive system, the respiratory system, bones and joints (e.g., bony metastases), soft tissue, the skin (e.g., melanoma and squamous cell carcinoma), breast, the genital system, the urinary system, the eye and orbit, the brain and nervous system (e.g., glioma, glioblastoma, and neuroblastoma), or the endocrine system (e.g., thyroid) and is not necessarily the primary tumor. Indeed, the tumor can be a metastasis of a primary tumor located in a different tissue or organ. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, non-small cell lung carcinoma. Tumors can arise in the uterine cervix, uterine corpus, ovary, vulva, vagina, prostate, testis, and penis, which make up the male and female genital systems, and the urinary bladder, kidney, renal pelvis, and ureter, which comprise the urinary system. Preferably, the cancer to be treated is selected from the group consisting of breast cancer, ovarian cancer, non-small cell lung carcinoma, and colorectal carcinoma.

The cancer also can be a hematologic malignancy. In this regard, the cancer can be located in lymphatic or hematopoietic tissues. For example, the cancer can be a lymphoma (e.g., Hodgkin's disease, Non-Hodgkin's lymphoma, and Burkitt's and Burkitt-like leukemia/lymphoma), multiple myeloma, leukemia (e.g., acute lymphocytic leukemia, chronic lymphocytic leukemia, acute myeloid leukemia, chronic myeloid leukemia, blast phase chronic myeloid leukemia, and the like), or high-risk myelodysplastic syndrome. Descriptions of hematologic malignancies and methods of treatment therefor are disclosed in U.S. Patent Application Publication No. 2002/0193425.

The cancer can be subjected to different therapies. In this regard, the inventive method is useful in treating cancers (i.e., destruction of tumor cells or reduction in tumor size) that have been proven to be resistant to other forms of cancer therapy, such as radiation-resistant tumors. The cancer or tumor also can be of any size. Ideally, in treating the mammal for cancer, the inventive method results in cancerous (tumor) cell death and/or reduction in tumor size. It will be appreciated that tumor cell death can occur without a substantial decrease in tumor size due to, for instance, the presence of supporting cells, vascularization, fibrous matrices, etc. Accordingly, while reduction in tumor size is preferred, it is not required in the treatment of cancer.

The cancer or tumor can be amenable to surgical removal (i.e., “resection”). In this respect, the inventive method can be used following surgical resection to eliminate any residual tumor cells. Alternatively, the tumor can be surgically unresectable. In this case, the inventive method can be used to effect shrinkage of the tumor, thereby facilitating surgical resection.

An “effective amount” means an amount sufficient to show a meaningful benefit in an individual, e.g., promoting at least one aspect of tumor cell cytotoxicity, or treatment, healing, prevention, or amelioration of other relevant medical condition(s) associated with a particular cancer. The dose will be determined by the potency of the particular compound employed for treatment, the severity of the cancer to be treated, as well as the body weight and age of the individual. The size of the dose also will be determined by the existence of any adverse side effects that may accompany the use of the particular compound employed. It is always desirable, whenever possible, to keep adverse side effects to a minimum.

The dosage can be in unit dosage form, such as a tablet or capsule. The term “unit dosage form” as used herein refers to physically discrete units suitable as unitary dosages for human and animal subjects, each unit containing a predetermined quantity of a compound, alone or in combination with other active agents, calculated in an amount sufficient to produce the desired effect in association with a pharmaceutically acceptable diluent, carrier, or vehicle. The specifications for the unit dosage forms of the present invention depend on the particular embodiment employed and the effect to be achieved, as well as the pharmacodynamics associated with each compound in the host. The dose administered should be an effective amount, i.e., an amount effective to be cytotoxic against the cancer cells to be treated.

Since the “effective amount” is used as the preferred endpoint for dosing, the actual dose and schedule can vary, depending on interindividual differences in pharmacokinetics, drug distribution, and metabolism. The “effective amount” can be defined, for example, as the blood or tissue level desired in the patient that corresponds to a concentration of one or more compounds according to the invention. The “effective amount” for a given compound of the present invention also can vary when the composition of the present invention comprises another active agent or is used in combination with another composition comprising another active agent.

One of ordinary skill in the art can easily determine the appropriate dose, schedule, and method of administration for the exact formulation of the composition being used, in order to achieve the desired “effective amount” in the individual patient. One skilled in the art also can readily determine and use an appropriate indicator of the “effective amount” of the compound of the present invention by pharmacological end-point analysis.

Generally, an amount of a present inventive compound up to about 200 mg/kg body weight, preferably from about 0.5 mg/kg body weight to about 50 mg/kg body weight (e.g., about 1 mg/kg, about 10 mg/kg, about 20 mg/kg, about 30 mg/kg, or about 40 mg/kg) is preferred, especially from about 1 mg/kg body weight to about 20 mg/kg body weight (e.g., about 5 mg/kg, about 8 mg/kg, about 12 mg/k, about 15 mg/kg, or about 18 mg/kg). In certain applications, multiple daily doses are preferred. Moreover, the number of doses will vary depending on the means of delivery and the particular compound administered.

In some embodiments, it may be advantageous to employ a method of administering the inventive compound wherein a dose is continuously administered to the patient over a prolonged period of time. For example, continuous infusion of the patient with the inventive composition the may be desirable. In this regard, the duration of the administration of the dose of the inventive composition may be any suitable length of time. Standard infusion rates for chemotherapeutics agents are known in the art and can be used in the inventive method, or modified in any suitable manner according to the nature of the disease. Aspects of cancer chemotherapy and dosing schedules are described in, for example, Bast et al. supra.

The inventive method can be performed in combination with other anti-cancer therapeutic methods to achieve a desired biological effect in a patient. In one embodiment, radiation therapy can be used before, during, or after the inventive method as an adjuvant method for further reducing the size of one or more tumors in the mammal. Any type of radiation can be administered to a patient, so long as the dose of radiation is tolerated by the patient without significant negative side effects. Suitable types of radiotherapy include, for example, ionizing (electromagnetic) radiotherapy (e.g., X-rays or gamma rays) or particle beam radiation therapy (e.g., high linear energy radiation). In another embodiment, the inventive composition is administered before, during, or after surgical resection of a tumor. Complete surgical removal of tumor tissue is often complicated by invasion of the tumor tissue into surrounding tissues and indefinite margins of the mass. As described herein, treatment of a tumor using the inventive method leads to tumor shrinkage, which will facilitate resection. Ideally, surgical resection of a tumor is performed after completion of the inventive method. Surgical resection of a tumor can be performed at any time after completion of the inventive method, so long as the patient is allowed sufficient time to recover from the administration of the inventive composition.

The following examples further illustrate the invention but, of course, should not be construed as in any way limiting its scope.

### EXAMPLES

Reaction progress was monitored with the use of analytical thin layer chromatography (TLC). High performance liquid chromatography (HPLC) analyses were typically performed using a 10-95% gradient of water-acetonitrile over 25 min on a Phenomenex Luna C18 column. ¹H NMR and ¹³C NMR (nuclear magnetic resonance) spectra were determined on Varian Inova 400 and 500 or VXR-300 spectrometers. Infrared. (IR) spectra were determined on a Perkin Elmer Paragon FT-IR spectrophotometer. All synthetic procedures, except for hydrolyses, were performed under positive pressure of dry nitrogen.

### Example 1

This example illustrates the synthesis of 3-tert-butyldimethylsilyl (TBDMS) bruceolide starting from a mixture of bruceolide esters isolated from a natural source.

The mixture of bruceolide esters (175 g, 335.9 mmol) and TBDMS-Cl (152.3 g, 3 eq) were dissolved in DMF (1.75 L). Imidazole (75.5 g, 3.3 eq) was added to the solution, and the contents were stirred for 17 hours at ambient temperature. The reaction was quenched by pouring the solution into a mixture of isopropanol (2 L) and water (1.75 L) in a separatory funnel. After vigorous shaking, the phases were allowed to separate and the aqueous lower phase withdrawn. The separated organic layer was washed with saturated ammonium chloride (1 L). The organic layer was then washed with water (3×1 L). The solvent was removed on a rotary evaporator, and a solvent exchange was performed with heptanes (1 L). The residue was dissolved in toluene (750 mL) and the solvent removed under vacuum. The residue was re-dissolved in toluene (1.1 L) and most of the solvent removed. Heptanes (1.5 L) were slowly added to the oil while continuing to rotate the flask on the rotavap. The flask and contents were rotated on the rotavap for 30 minutes at 40° C., then the flask was removed from the water bath and the resulting heterogeneous mixture was allowed to rotate overnight at ambient temperatures to maximize precipitation of the product. The solid product was collected by filtering the mixture through Whatman #4 filter paper in a 19 cm Buchner funnel under vacuum and washed with heptanes (500 mL). The solid material was placed in a vacuum oven for 19 hours at 60° C. The yield was 186.3 g (87.3%).

The TBDMS-bruceolide ester mixture (25 g, 39.37 mmol, molecular equivalents calculated using the molecular weight of TBDMS-brusatol) was dissolved in THF (75 mL). A solution of potassium methoxide in methanol (1.5M, 79 mL, assuming 100% KOMe was added to the methanol, which it is not) was added all at once. The reaction was allowed to stir for 2 hours at ambient temperature and then quenched by adding slowly (over 9 minutes) to a mixture of water (250 mL), heptanes (250 mL) and citric acid (7.5 g, 1 eq) at ambient temperature. This mixture was stirred for 15 minutes and then placed in the refrigerator (˜4° C.) for 1 hour. The mixture was filtered, collecting the solid on Whatman #4 paper in an 11 cm Buchner funnel and the solid washed with water (300 mL). The collected solid was dried in the vacuum oven at 50° C. overnight. The yield was 15.97 g of solid, which was 75.9 wt % of TBDMS-bruceolide by HPLC.

The TBDMS-bruceolide solid (15.97 g) was placed in a fluted funnel and warm acetone was added and mixed with the solid using a spatula. The acetone was allowed to drip through the filter while adding additional warm acetone and mixing. The resulting solution was concentrated to dryness. The yield was 14.14 g with 82.7 wt % and 82.2 area % by HPLC.

### Example 2

This example illustrates the synthesis of 15-O-(3′-hydroxy-3′-isopropyl-4′-methylpentanoyl)bruceolide.

### Step A: Preparation of 3-hydroxy-3-isopropyl-4-methylpentanoic acid (3,3-diisopropyl-β-hydroxypropanoic acid)

To lithium diisopropylamide (30 wt % suspension in mineral oil) (124 g=37.2 g amide, 347 mmol), suspended in THF (175 mL, −65° C.) under nitrogen, was added acetic acid (99.99%, 12 mL=12.58 g, 209 mmol) over 15 min. Upon stirring for ½ hr. (−65° C.), the reaction mixture was slowly warmed to rt. and stirred at the THF boiling temperature for 2.5 hr. After additional stirring (½ hr) in a hot water bath, the mixture was gradually cooled to −60° C. and 2,4-dimethyl-3-pentanone (23.5 mL, 165 mmol) was injected over 30 min. The mixture was then stirred over a water-bath with a slow increase in temperature (to −35° C.) and was placed overnight in a freezer (−20° C). Upon removal from the freezer, the reaction mixture was warmed to rt., stirred (4 hr), quenched with a 1:1 w/w ice-water mixture (1.1 kg) and extracted twice with toluene (1×350 mL, 1×250 mL). The combined toluene extracts were washed with cold water (200 mL). The combined water phases were placed in an ice-water bath, acidified with 4N HCl (to pH 1-2) and the still cold emulsion was extracted with rapid stirring/shaking with MTBE (2×300 mL and 1×500 mL). The combined MTBE extracts were washed with brine (350 mL), dried overnight over anhyd. Na₂SO₄, filtered, and evaporated, to afford, after drying (3 hr., standard vacuum), 16.68 g (58.1%) of the product as a light brown powder, satisfactory for use without further purification.

The structure was supported by ¹H NMR and IR analyses.

### Step B: Preparation of 15-O-(3′-hydroxy-3′-isopropyl-4′-methylpentanoyl)-3-(t-butyldimethylsilyl)bruceolide

3-TBDMS-bruceolide (25.07 g, 0.0454 mol), 3-hydroxy-3-isopropyl-4-methylpentanoic acid (12.13 g, 0.0697 mol) and 4-pyrrolidinopyridine (10.35 g, 0.0699 mol) were placed in a RB flask equipped with a stirring bar. The flask was flushed with dry nitrogen; dichloromethane (750 mL) was injected; stirring of the reaction mixture resulted in a clear solution. The flask was placed in an ice-water bath (0-2° C.) and a solution of DCC in dichloromethane (1.4M, prepared by dissolution of 14.5 g of solid DCC in dichloromethane and adjusting the volume to 50 mL) (40 mL, 56 mmol) was injected dropwise into the reaction mixture over 1 hr. While remaining in the ice-water bath, the reaction mixture was stirred 15 hr. during which the bath temperature slowly rose to 17° C. The bath temperature was then increased (17° C. to 35° C.) and stirring was continued (3 hr.) The reaction mixture was filtered from DCU, concentrated to ca. ⅓ the previous volume and loaded onto a silica gel 60 column (EM Sciences 9385-9; bed size 5×30.5 cm, preconditioned in toluene). The load was washed with dichloromethane (1×500 mL, 1×1L) and eluted with a gradient 5-45% v/v EtOAc/dichloromethane solvent mixture with 5% intervals. Upon concentration of the collected fractions afforded the purified product yielding 22.07 g (68.3%).

### Step C: Preparation of 15-O-(3′-hydroxy-3′-isopropyl-4′-methylpentanoyl)bruceolide

15-O-(3′-Hydroxy-3′-isopropyl-4′-methylpentanoyl)-3-TBDMS-bruceolide (10.62 g, 15 mmol) was dissolved at rt. (22° C.) with stirring in a 1:2 v/v EtOAc/dichloromethane solvent mixture (450 mL) and 0.65M TBAF solution in THF containing ca. 5% water (prepared by dissolving TBAF (10 g) in THF (55 mL), adding ca. 2 mL water and shaking to obtain a clear solution with total volume adjusted to 58 mL) was added with pH control (pH 5-8). The deprotection was judged complete (5.5-6 hr.) by TLC. The entire reaction volume was loaded on a silica gel (EM Sciences 9385-9) column (bed size 5×34 cm, preconditioned in toluene, washed with an equal volume of dichloromethane (450 mL)) and eluted with gradient EtOAc/dichloromethane (20%-65% with intervals of 10% and ca. 1.2 L portions). Combined fractions containing pure product afforded, after evaporation and standard drying, 7.56 g (84.1% yield) of product material (purity=99.28%, UV detector=280 nm).

IR (Nujol, cm⁻¹) 3300, 1750, 1665, and 1635. ¹H NMR (CDCl₃) δ 6.40 (m, 1H, H-15), 6.10 (s, 1H, enol-OH), 4.75 (m, 1H, H-7), 4.72 (d, 1H, H-20), 4.25 (m, 1H, H-11), 4.22 (s, 1H, H-12), 3.85 (s, 3H, H-22), 3.80 (d, 1H, H′-20), 3.05 (d, 1H, H-14), 2.95 (s, 1H, H-1), 2.92 (m, 1H, H-6), 2.48 (d, 2H, H-2′), 2.42 (s, 1H, H′-1), 2.38 (m, 1H, H-5), 2.15 (m, 1H, H-9), 1.99 (m, 2H, H-4′, 4″), 1.85 (s, 3H, H-18), 1.78 (m, 1H, H′-6), 1.40 (s, 3H, H-19), 0.90-1.00 (multiple d, 12H, H-5: CH3's). ¹³C NMR (CDCl₃) δ 48.53 (C-1), 192.02 (C-2), 144.06 (C-3), 127.69 (C-4), 41.85 (C-5), 29.03 (C-6), 82.61 (C-7), 45.63 (C-8), 41.98 (C-9), 41.08 (C-10), 71.02 (C-11), 75.63 (C-12), 81.38 (C-13), 51.60 (C-14), 66.57 (C-15), 166.61 (C-16), 13.35 (C-18), 15.43 (C-19), 171.94 (C-21), 73.96 (C-20), 53.21 (C-22), 172.18 (C-1′), 35.68 (C-2′), 76.92 (C-3′), 34.84 (C-4′), 34.78 (C-4″), 17.56-17.52 (4C-5's).

### Example 3

This example illustrates the synthesis of 15-O-(3′-isopropyl-4′-methyl-2′-pentenoyl)-bruceolide.

### Step A: Synthesis of 15-O-(3′-isopropyl-4′-methyl-2′-pentenoyl)-3-TBDMS-bruceolide

3-Hydroxy-3-isopropyl-4-methylpentanoic acid (1,190.0 mg, 6.83 mmol) was additionally dried before use at standard vacuum for 30 min. It was combined with 4-pyrrolidinopyridine, (802 mg, 5.41 mmol); the flask was purged with nitrogen, and anhydrous toluene (120 mL) was injected. The reaction mixture was placed in an ice-water bath (0-2° C.) and 1,3-dicyclohexylcarbodiimide (DCC) (1.0 M solution in methylene chloride, 7.5 mL, 7.5 mmol) was added dropwise over 30 min. The reaction mixture was left stirring overnight in the bath. The next day, the bath temperature (17° C.) was raised to 30° C. and the reaction mixture was stirred at that temperature for two hr. The reaction mixture was filtered from dicyclohexylurea (DCU) (after drying 1,521.0 mg DCU, 90% conversion of DCC into DCU). The filtrate, containing mainly 3-isopropyl-4-methyl-2-pentenoic acid, and in part, its active ester, was concentrated to a volume ca. 5-7 mL. The flask was purged with a nitrogen stream and methylene chloride (110 mL) was added. This was followed by the addition of 4-pyrrolidinopyridine, (208 mg, 1.4 mmol) and 3-TBDMS-bruceolide (2,635.0 mg, 4.77 mmol). The reaction mixture was stirred at room temperature for two hr and a second portion of DCC (1.0 M solution in dichloromethane, 7 mL, 7 mmol) was added in two portions: the first portion (5 mL) was added dropwise over a period of 10 min, and the second portion (remaining 2 mL) was added dropwise two hr later. The reaction mixture was left stirring overnight in an ice-bath. The next day, the bath temp. (17° C.) was raised to ca. 25-30° C. and stirring of the mixture was continued for the next two hr. The reaction mixture was filtered from the DCU, and the filtrate was subjected to normal phase chromatography (silica 1092 with gradient elution with 0-15% v/v EtOAc/CH₂Cl₂, followed by isocratic elution with 50% v/v EtOAc/toluene) and separated/purified to afford 1,269.4 mg (38.5% total yield) of 15-O-(3′-isopropyl-4′-methyl-2′-pentenoyl)-3-TBDMS-bruceolide. Rf=0.60, 4:1, v/v, EtOAc/heptane. LCMS SSQ: RT=22.18 min., purity=97.8%, M+NaCH₂CN=753 (100%) for MW=690.

### Step B: Preparation of 15-O-(3′-isopropyl-4′-methyl-2′-pentenoyl)-bruceolide

15-O-(3′-Isopropyl-4′-methyl-2′-pentenoyl)-3-TBDMS-bruceolide (1,171.3 mg, 1.7 mmol) was dissolved in a 6:4, v/v, CH₂Cl₂/EtOAc solvent mixture (100 mL). A 0.2M solution of tetrabutylammonium fluoride in THF was prepared by the 5× dilution with THF of the Aldrich commercial 1.0 M solution in THF. This solution (7.5 mL, 1.5 mmol) was added dropwise to the mixture while maintaining a pH range of 7-8. The reaction was complete within 4 hr. Rf=0.41, 4:1 v/v EtOAc/heptane. LCMS SSQ 2 mm. meth. RT=13.64 min., peak area=100%, M+Na=599, M+NaCH₂CN=639, calc. C₃₀H₄₀O₁₁: M⁺=576.

The reaction mixture was transferred onto a normal phase chromatographic column (silica 1092 with gradient elution with EtOAc/dichloromethane) to afford, after separation/purification, 969.2 mg (91.5% yield) of the 15-O-(3′-isopropyl-4′-methyl-2′-pentenoyl)-bruceolide.

### Step C: Crystallization 15-O-(3′-isopropyl-4′-methyl-2′-pentenoyl)-bruceolide

The complete batch of 15-O-(3′-isopropyl-4′-methyl-2′-pentenoyl)-bruceolide (969.2 mg) was placed with stirring in absolute ethyl alcohol (30 mL) at the water bath temperature of 70° C. After 15 min of stirring in the bath, the batch, still containing partially insoluble material, was filtered. The filtrate was concentrated to ca. half of the previous volume and stored in a freezer (−5 to−11° C.) for 10 days. Filtration and drying at standard vacuum afforded 681.1 mg (crystallization yield 70.2%) of the product, Mp=225-230° C.

### Example 4

This example illustrates the synthesis of additional compounds of formula (I), (II), (III), or (IV).

Non-commercially available bruceolide was prepared by extraction from a natural source (i.e., Brucea javanica (L.) Merr.). Since the ring systems of formulae (I), (II), (III) and (IV) contain several alcoholic functions, to achieve proper regioselectivity for the ester formation at position 15, the starting ring system has to be sequentially protected at the more nucleophilic center(s) present, the C-15 esterification executed and the product deprotected at the final stage.

3-TBDMS-bruceolide was prepared as described in Example 1. The 3-TBDMS mixed esters were hydrolyzed at position 15 to afford the desired protected intermediate, selectively esterified as described in Examples 2 and 3 and deprotected to provide the compounds listed in Table 1.

In a specific example, the following procedure was used.

To a cooled stirred solution of 3-hydroxy-3-isopropyl-4-methylpentanoic acid (100 mg, 0.57 mmol) in anhydrous dichloromethane (1 mL) held under N₂ was added anhydrous pyridine (0.4 mL, 9 eq). The reaction mixture was cooled over an ice/brine bath for 15 min when deoxofluor (0.28 mL, 2.5 eq) was added dropwise with stirring. After 15 min the reaction solution was removed from the bath and stirring continued for 5 hr when dichloromethane (6 mL) was added. The reaction was quenched with water (4 mL), stirred, and transferred to a separatory funnel. The layers were separated, and the organic layer was dried over Na₂SO₄, concentrated to less than 0.3 mL, and diluted with dichloromethane (0.6 mL) to give the acid fluoride to be used directly in the next step of the sequence.

To a suspension of 3-O-TBDMS-bruceolide (150.2 mg, 0.27 mmol,) and 4-pyrrolidinopyridine (80 mg, 0.54 mmol) in dichloromethane (0.4 mL) was added the acid fluoride from the previous reaction. The reaction mixture stirred for 16 hr when analysis by HPLC indicated the absence of starting material and the formation of the desired unprotected coupled product, as well as the protected coupled product.

TBAF (1 mL, 1 mmol) was added to the reaction mixture with stirring (1 hr) to deprotect the protected coupled product to give unprotected coupled product, which was purified by column chromatography followed by alcohol crystallization.

Using a similar procedure, the corresponding bruceolide derivatives were prepared from 2-cyclohexylidineacetic acid, 2-cyclopentylidineacetic acid, and 3-ethylpent-2-enoic acid.

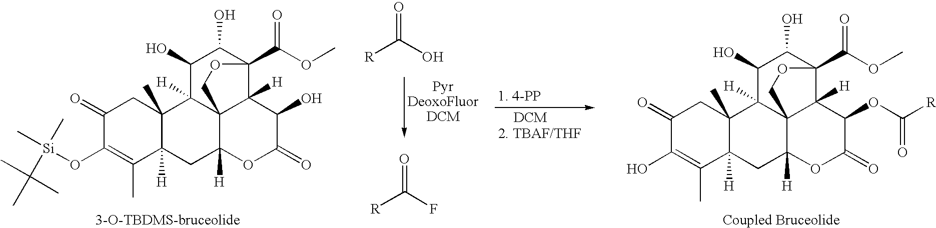

TABLE 1

| | Form. | | | | | | |
|---|---|---|---|---|---|---|---|
| Entry | # | R | R₁ | R₂ | R₃ | R₄ | R₅ |
| 1 | I | H | H | H | (CH₂)₅CH═CH(CH₂)₇CH₃ | H | H |
| 2 | I | H | H | H | CH₂CH₂CH═CH₂ | H | H |
| 3 | I | H | H | H | ⁿPr | H | H |
| 4 | I | H | CH₃ | CH₃ | CH₃ | H | H |
| 5 | I | H | CH₃ | CH₃ | OH | H | H |
| 6 | I | H | -cyclopentyl- | | OH | H | H |
| 7 | I | H | -cyclohexyl- | | OH | H | H |
| 8 | I | H | Et | Et | OH | H | H |
| 9 | I | H | H | Ph | —NHC(O)Ph | H | OH |
| 10 | I | H | Et | ⁱPr | OH | H | H |

| | | R | R₁₀ | R₁₁ | R₁₂ |
|---|---|---|---|---|---|
| 11 | II | H | -1-cyclopentenyl- | | — |
| 12 | II | H | -1-cyclohexenyl- | | — |
| 13 | II | H | -2′,5′-dimethyl- | | — |
| | | | 3′-furanyl- | | |
| 14 | II | H | H | H | Ph |
| | | R | R₁₈ | R₁₉ | R₂₀ |
| 15 | III | H | H | H | Ph |
| 16 | III | H | H | Et | Et |
| 17 | III | C(O)CH═C(Me)ⁱPr | H | Me | ⁱPr |

| | | | | |
|---|---|---|---|---|
| 18 | III | H | H | -cyclohexyl- |
| 19 | III | H | H | -cyclopentyl- |

| | | R | R₂₆ |
|---|---|---|---|
| 20 | IV | H | 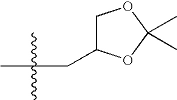 |
| 21 | IV | 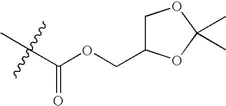 | 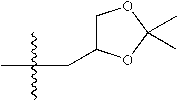 |
| 22 | IV | H | CH₂CH═CH₂ |
| 23 | IV | CO₂CH₂CH═CH₂ | CH₂CH═CH₂ |

### Example 5

This example demonstrates a method of killing tumor cells by administering an inventive compound to tumor cells in vitro.

The following human tumor cell lines were obtained and cultured in sextuplicate microwells at 37° C. and 5% CO₂ in humidified air and in 10% FBS in Eagles MEM: SK—N—FI, SK—N-AS, LS-174T, WiDr, HuTu-80, MCF7, NCI-AR, MES-Sar, and PL-45. Cells from each cell line were separately treated with an amount of one of the compounds of Formula I, II, or III (see Table 1). Cells were exposed to each compound (0.1 μg/mL or 1.0 μg/mL) for one hour or 24 hours, rinsed with drug-free media, and incubated for an additional five days. The number of viable cells after five days was measured using an MTT (3-(4,5-dimethylthiazol-2-yl)-2,5-diphenyltetrazolium bromide) assay (see, e.g., Mosmann, J. Immunol. Methods, 65(1-2), 55-63 (1983)).

The cytotoxicity of each drug was determined and expressed as the percent of cells killed (“% kill”). The results of this analysis are set forth in Tables 2 and 3.

TABLE 2

| | | | | Cell Line | |
|---|---|---|---|---|---|
| | | | | (% Kill) | |
| | Dole | | | SK-N- | LS- |
| Compound | (μg/mL) | Exposure | | AS | 174T |
| 15-O-(cis-9′- | 1.0 | 1 | h | 0% | 4% |
| octadecenoyl)-bruceolide | | 24 | h | 0% | 13% |
| 15-O-cinnamyl-bruceolide | 1.0 | 1 | h | 0% | 16% |
| | | 24 | h | 61% | 64% |
| 15-O-(1′-cyclopentenoyl)- | 1.0 | 1 | h | 0% | 0% |
| bruceolide | | 24 | h | 0% | 8% |
| 15-O-(1′-cyclohexenoyl)- | 1.0 | 1 | h | 0% | 12% |
| bruceolide | | 24 | h | 5% | 35% |
| 15-O-(2′,5′-dimethyl-3′- | 1.0 | 1 | h | 0% | 12% |
| furoyl)-bruceolide | | 24 | h | 34% | 53% |
| 15-O-phenylacetyl | 1.0 | 1 | h | 0% | 20% |
| bruceolide | | 24 | h | 38% | 61% |
| 15-O-hexanoylbruceolide | 1.0 | 1 | h | 0% | 19% |
| | | 24 | h | 41% | 57% |
| 15-O- | 1.0 | 1 | h | | 13% |
| (cyclohexylideneacetyl)- | | 24 | h | | 63% |
| bruceolide | | | | | |
| 15-O-(3′,3′- | 1.0 | 1 | h | 0% | 7% |
| dimethylbutanoyl)- | | 24 | h | 62% | 60% |
| bruceolide | | | | | |
| 15-O-(3′-ethyl-2′-pentene- | 1.0 | 1 | h | 0% | 24% |
| oyl)-brucelide | | 24 | h | 66% | 64% |
| 15-O-(3′-hydroxy-3′- | 1.0 | 1 | h | | 29% |
| methylbutanoyl)- | | 24 | h | | 64% |
| bruceolide | | | | | |
| 15-O-[(1′- | 1.0 | 1 | h | | 36% |
| hydroxycyclopentyl)- | | 24 | h | | 65% |
| acetyl]-bruceolide | | | | | |
| 15-O-[(1′- | 1.0 | 1 | h | 16% | 23% |
| hydroxycyclohexyl)- | | 24 | h | 80% | 62% |
| acetyl]-bruceolide | | | | | |
| 15-O-(3′-ethyl-3′- | 1.0 | 1 | h | 6% | 10% |
| hydroxypentanoyl)- | | 24 | h | 71% | 50% |
| bruceolide | | | | | |
| 15-O- | 1.0 | 1 | h | 0% | 0% |
| (cyclopentylideneacetyl)- | | 24 | h | 50% | 50% |
| bruceolide | | | | | |
| 15-O-(allyloxycarbonyl)- | 1.0 | 1 | h | 1% | 1% |
| bruceolide | | 24 | h | 43% | 43% |
| 12,15-O,O-bis- | 1.0 | 1 | h | 0% | |
| (allyloxycarbonyl)- | | 24 | h | 2% | |
| bruceolide | | | | | |
| 15-O- | 1.0 | 1 | h | 0% | |
| (solketyloxycarbonyl)- | | 24 | h | 28% | |
| bruceolide | | | | | |
| 12,15-O,O- | 1.0 | 1 | h | 3% | |
| bis(Solketyloxycarbonyl)- | | 24 | h | 5% | |
| bruceolide | | | | | |
| 12-O-(3,4-dimethyl-2- | 1.0 | 1 | h | 0% | |
| pentenoyl)-bruceantin | | 24 | h | 0% | |
| (+/−)-15-O-(3′-ethyl-3′- | 0.1 | 1 | h | 0% | 0% |
| hydroxy-4′-methyl)- | | 24 | h | 0% | |
| bruceolide | 1.0 | 1 | h | 0% | 0% |
| | | 24 | h | 0% | |
| 15-O-(3′-hydroxy-3′- | 0.1 | 1 | h | 0% | 0% |
| isopropyl-4′- | | 24 | h | | |
| methylpentanoyl) | 1.0 | 1 | h | 3% | 13% |
| bruceolide | | 24 | h | | |

TABLE 3

| | | | Cell Line | | | | | |
|---|---|---|---|---|---|---|---|---|
| | Dose | | | HuTu- | | NCI- | MES | |
| Compound | (μg/mL) | Exposure | WiDr | 80 | MCF7 | AR | Sar | PL-45 |
| (+/−)-15-O- | 0.1 | 1 h | 0% | | | 0% | 0% | |
| (3′-ethyl-3′- | | 24 h | | | | | | |
| hydroxy-4′- | 1.0 | 1 h | 0% | | | 0% | 0% | |
| methyl)- | | 24 h | | | | | | |
| bruceolide | | | | | | | | |
| 15-O-(3′- | 0.1 | 1 h | 0% | 0% | 12% | 0% | 0% | 0% |
| hydroxy-3′- | | 24 h | | | | | | |
| isopropyl-4′- | 1.0 | 1 h | 0% | 7% | 31% | 0% | 0% | 0% |
| methylpentan | | 24 h | | | | | | |
| oyl) | | | | | | | | |
| bruceolide | | | | | | | | |
| 15-O-(3′- | 0.1 | 1 h | 0% | | | 0% | 0% | |
| hydroxy-3′- | | 24 h | | | | | | |
| isopropyl-4′- | 1.0 | 1 h | 23% | | | 0% | 0% | |
| methylpentan | | 24 h | | | | | | |
| oyl) | | | | | | | | |
| bruceolide | | | | | | | | |

### Example 6

This example demonstrates the in vitro toxicity of the inventive compounds in solid tumor cells as compared to leukemia cells.

A disk diffusion assay was performed to define the differential cell killing ability of 15-O-(3′-hydroxy-3′-isopropyl-4′-methylpentanoyl)bruceolide. Seven cell types were assayed: L1210 (ATCC CCL-219) (murine leukemia), CCRF-CEM (ATCC CCL-119) (human leukemia), Colon 38 (murine colon adenocarcinoma), HCT116 (ATCC CCL-247) (human colorectal carcinoma), H125 (human lung carcinoma). The murine and human granulocyte/macrophage progenitor cell line (CFU-GM), served as controls.

Colon 38 cells (about 1 gram) were cut into small fragments in 15 mL of Hank's Balanced Salt Solution (HBSS) over a 100-mesh sieve and gently forced through by the scissors with HBSS constantly perfusing the sieve. The resulting material was then drawn into and out of a 5 mL syringe without a needle to further disperse the cell clumps. The cell solution was diluted and plated in 0.3% agarose in DMEM plus 10% heat-inactivated Bovine Calf Serum (BCS). For plating of all of the cell types (except controls), the 60 mm plates were first prepared with a hard agar bottom layer (0.6% agar in RPMI-1640 plus 15% BCS).

HCT116 and H125 cells were maintained in cell culture, and were removed from culture by a trypsin-collagenase-DNAase cocktail. The plating efficiencies of each cell type was sufficiently high that 30,000 cells in 3 mL produced the desired number of colonies (i.e., over 10,000 per plate) in 60 mm plates. This soft agar top layer (0.3% with the serum and media as above) plus the titrated tumor cells were poured into the plates and allowed to solidify.

For CFU-GM cells, the femoral marrow of BD2F1 mice was flushed with MEM alpha (2 mL per femur). The cells were passed through an 18 gauge needle twice and the monodispersed suspension was counted. A total of 1.5×10⁶ cells were plated in 3 mL of 0.3% agar with the addition of 10% L cell conditioned media, which provided colony stimulating factor, in MEM-alpha plus 10% BCS. For human CFU-GM, the cells were obtained from Poietic Technologies, Inc. (Gaithersburg, Md.) and washed twice with PBS before being titered and added to the agar mixture. The same cell number, culture conditions, and conditioning factors used with respect to the murine CFU-GM cells were used with the human cells, except that the cultures were assessed after 10 days of incubation.

Powder samples of 15-O-(3′-hydroxy-3′-isopropyl-4′-methylpentanoyl)bruceolide, 15-(3-isopropyl-4-methyl-2-pentenoyl)-bruceolide, and, for comparative purposes, peninsularinone, bruceantin, glaucarubolone, and taxol were solubilized and diluted in ethanol. A 15 μl sample of each compound was dropped onto a 6.5 mm disk (Baxter filter disks). The disks were allowed to dry overnight and then placed close to the edge of a petri dish. Depending upon the cell type, the dishes were incubated for 7 to 10 days, and examined by an inverted stereo-microscope (10×) for measurement of the zone of inhibition. The zone of inhibition was measured from the edge of the filter disk to the beginning of normal-sized colony formation. The diameter of the filter disk, 6.5 mm, was arbitrarily taken as a zone of 200 units. Compounds that were selective for killing solid tumors, as compared to leukemia or normal cells (“solid tumor selective”) were defined as those compounds which produced a difference in zones between solid tumor cells and either normal or leukemia cells of at least 250 units. The results of this analysis are set forth in Table 4, which illustrates the relative diameters of the zones of inhibition by various drugs on the growth of the respective cell types at various drug dilutions.

TABLE 4

| | | Cell Line | | | | | | |
|---|---|---|---|---|---|---|---|---|
| Compound | | | | | | | murine | human |
| (1 mg/mL) | Dilution | L1210 | Colon 38 | H-116 | H-125 M | CEM | CFU-GM | CFU-GM |
| DMSO | | 0 | 0 | 0 | 0 | 0 | 0 | 0 |
| | | >1000 | >1000 | >1000 | >1000 | | | |
| Bruceantin | 1/10 | 1000 | >1000 | 1000 | >1000 | | 550 | >1000 |
| (comparative) | 1/100 | 500 | 1000 | 400 | 600 | 450 | 200 | 650 |
| | 1/100 | 0 | 600 | | | | 150 | |
| | 1/1000 | 100 | 600 | 0 | 100 | | 0 | |
| | 1/1000 | 0 | 450 | | | | 0 | |
| 15-O-(3′-hydroxy-3′- | | 1000 | >1000 | >1000 | >1000 | | | |
| isopropyl-4′- | 1/10 | 550 | >1000 | 1000 | 950 | 650 | 50 | 900 |
| methylpentanoyl) | 1/10 | | | 900 | 50 | 700 | | |
| bruceolide | 1/100 | 200 | 750 | 450 | 250 | 100 | 300 | 600 |
| (inventive) | 1/100 | | 600 | 100 | 0 | 150 | | |
| | 1/100 | 200 | 500 | | | | 200 | |
| | 1/400 | 0 | 300 | | | | 100 | |
| | 1/1000 | 0 | 400 | 0 | 0 | | 0 | |
| | 1/1000 | 50 | 250 | | | | 0 | |
| 15-(3-isopropyl-4-methyl-2- | | >1000 | >1000 | >1000 | >1000 | | | |
| pentenoyl)-bruceolide | 1/10 | 1000 | >1000 | 750 | 1100 | 800 | 650 | >1000 |
| (inventive) | 1/100 | 650 | 850 | 100 | 500 | 400 | 350 | 550 |
| | 1/100 | 600 | 650 | | | | 600 | |
| | 1/1000 | 150 | 450 | 0 | 100 | | 0 | |
| Peninsularinone | | >1000 | >1000 | >1000 | >1000 | >1000 | >1000 | |
| (comparative) | 1/10 | >1000 | >1000 | >1000 | >1000 | >1000 | >1000 | |
| | 1/100 | 700 | >1000 | 950 | 1000 | 800 | 450 | |
| | 1/1000 | 300 | 800 | 450 | 400 | 500 | 0 | |
| | 1/1000 | 200 | 550 | | | | 100 | |
| | 1/4000 | 50 | 250 | | | | 0 | |
| | 1/10000 | 100 | 200 | | | | 0 | |
| Glaucarubolone | | 400 | >1000 | >1000 | >1000 | 900 | 50 | |
| (comparative) | 1/10 | 50 | >1000 | 600 | 700 | 600 | 0 | |
| | 1/10 | 0 | 600 | | | | 0 | |
| | 1/40 | 200 | 400 | | | | 0 | |
| | 1/40 | 0 | 400 | | | | 0 | |
| | 1/100 | 0 | 400 | 0 | 0 | 0 | 0 | |
| | 1/1000 | 0 | 0 | 0 | 0 | 0 | 0 | |
| Taxol | | 650 | >1000 | 850 | >1000 | >1000 | 650 | >1000 |
| (comparative) | 1/4 | 550 | 900 | 750 | 850 | >1000 | 600 | >1000 |
| | 1/16 | 500 | 750 | 650 | 750 | 900 | 550 | 800 |
| | 1/64 | 350 | 600 | 550 | 650 | 750 | 400 | 750 |
| | 1/256 | 50 | 450 | 400 | 500 | 550 | 150 | 550 |

This example demonstrates that the inventive compound is selectively cytotoxic to solid tumor cells, as compared to leukemic cells or non-tumor cells.

### Example 7

This example demonstrates the cytotoxic potency of the inventive compounds in solid tumor cells.

The degree of differential sensitivity of several inventive compounds was compared with respect to the Colon38 cell line, the L1210 cell line, and the control CFU-GM cell lines. The degree of differential sensitivity was estimated by calculating the difference between the zone of inhibition values, as determined in Example 6, for the solid tumor compared to either the leukemia (C38ΔL1210) or normal cell (C38ΔCFU).

The potency of each of several inventive compounds was determined by identifying the dilution required to yield an average zone of inhibition of 500 units for the Colon38 cell type. Then the potency and the zone differentials were multiplied to define a “product” (see Table 5), which can be used to rank the compounds tested for their potential for further development; the greater the product value the greater the probability of useful chemotherapeutic utility.

TABLE 5

| | | | | | |
|---|---|---|---|---|---|
| Compound | C38ΔL1210 | C38ΔCFU | Sum | Potency | Product |

Product > 4000

| | | | | | |
|---|---|---|---|---|---|
| 15-O-(3′-hydroxy-3′-isopropyl-4′- | 550 | 900 | 1450 | 256:5 | 7250 |
| methylpentanoyl)bruceolide | | | | | |
| (inventive) | | | | | |
| 15-(3-isopropyl-4-methyl-2- | 300 | 450 | 750 | 1000:6 | 4500 |
| pentenoyl)-bruceolide | | | | | |
| (inventive) | | | | | |
| 15-O-[(1′-hydroxycyclopentyl)- | 600 | 700 | 1300 | 64:4 | 5200 |
| acetyl]-bruceolide | | | | | |
| (inventive) | | | | | |
| 15-O-(2′,5′-dimethyl-3′-furoyl)- | 500 | 550 | 1050 | 64:4 | 4200 |
| bruceolide | | | | | |
| (inventive) | | | | | |
| 15-O-(cyclopentylideneacetyl)- | 350 | 450 | 800 | 256:5 | 4000 |
| bruceolide | | | | | |
| (inventive) | | | | | |
| 15-O-(allyloxycarbonyl)-bruceolide | 500 | 550 | 1050 | 64:4 | 4100 |
| (inventive) | | | | | |
| 15-O-phenylacetyl bruceolide | 500 | 500 | 1000 | 64:4 | 4000 |
| (inventive) | | | | | |
| Bruceantin | 500 | 550 | 1050 | 1000:6 | 6300 |
| (comparative) | | | | | |
| Peninsularinone | 450 | 650 | 1100 | 1000:6 | 6600 |
| (comparative) | | | | | |
| Glaucarubolone | 550 | 600 | 1150 | 64:4 | 4600 |
| (comparative) | | | | | |

3000 < Product < 4000

| | | | | | |
|---|---|---|---|---|---|
| 15-O-(3′-hydroxy-3′-isopropyl-4′- | 400 | 350 | 750 | 100:4.5 | 3375 |
| methylpentanoyl)bruceolide | | | | | |
| (inventive) | | | | | |
| 15-O-(allyloxycarbonyl) bruceolide | 350 | 250 | 600 | 1024:6 | 3600 |
| (inventive) | | | | | |
| (+/−)-15-O-(3′-ethyl-3′-hydroxy-4′- | 600 | 550 | 1150 | 16:3 | 3450 |
| methyl) bruceolide | | | | | |
| (inventive) | | | | | |
| 15-O-(3′-Ethyl-3′- | 350 | 500 | 850 | 64:4 | 3400 |
| hydroxypentanoyl bruceolide | | | | | |
| (inventive) | | | | | |
| 15-O-(3′,3′-dimethylbutanoyl) | 450 | 350 | 800 | 64:4 | 3200 |
| bruceolide | | | | | |
| (inventive) | | | | | |
| 15-O-[(1′-hydroxycyclohexyl)- | 300 | 450 | 750 | 16:3 | 3000 |
| acetyl] bruceolide | | | | | |
| (inventive) | | | | | |
| Peninsularinone | 250 | 300 | 550 | 2048:6.5 | 3575 |
| (comparative) | | | | | |
| Glaucarubolone | 400 | 400 | 800 | 100:4.5 | 3600 |
| (comparative) | | | | | |
| Taxol | 400 | 300 | 700 | 256:5 | 3500 |
| (comparative) | | | | | |

2000 < Product < 3000

| | | | | | |
|---|---|---|---|---|---|
| 15-O-(3′-hydroxy-3′- | 400 | 550 | 950 | 16:3 | 2850 |
| methylbutanoyl)bruceolide | | | | | |
| (inventive) | | | | | |
| 15-O- | 350 | 350 | 700 | 64:4 | 2800 |
| (cyclohexylideneacetyl)bruceolide | | | | | |
| (inventive) | | | | | |
| 12,15-O,O- | 400 | 600 | 1000 | 4:2 | 2000 |
| bis(Solketyloxycarbonyl)bruceolide | | | | | |
| (inventive) | | | | | |

1000 < Product < 2000

| | | | | | |
|---|---|---|---|---|---|
| 15-O-(solketyloxycarbonyl)- | 400 | 550 | 950 | 4:2 | 1900 |
| bruceolide | | | | | |
| (inventive) | | | | | |

Product < 1000

| | | | | | |
|---|---|---|---|---|---|
| 15-O-(1′-cyclopentenoyl)- | 350 | 400 | 750 | 1:1 | 750 |
| bruceolide | | | | | |
| (inventive) | | | | | |
| 15-O-hexanoylbruceolide | 400 | 200 | 600 | 1:1 | 600 |
| (inventive) | | | | | |
| 12,15-O,O-bis(allyloxycarbonyl)- | 300 | 350 | 650 | 1:1 | 650 |
| bruceolide | | | | | |
| (inventive) | | | | | |
| 15-O-(1′-cyclohexenoyl)- | 200 | 350 | 550 | 1:1 | 550 |
| bruceolide | | | | | |
| (inventive) | | | | | |

This example demonstrates that the inventive compounds are selectively potent against solid tumor cells.

### Example 8

This example demonstrates a method of killing human colon cancer cells using the inventive compound.

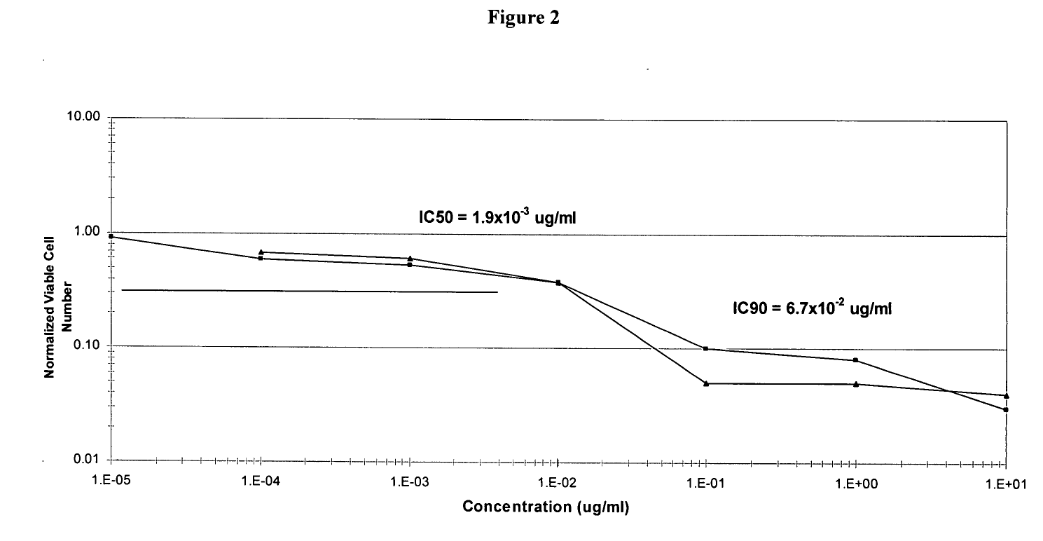

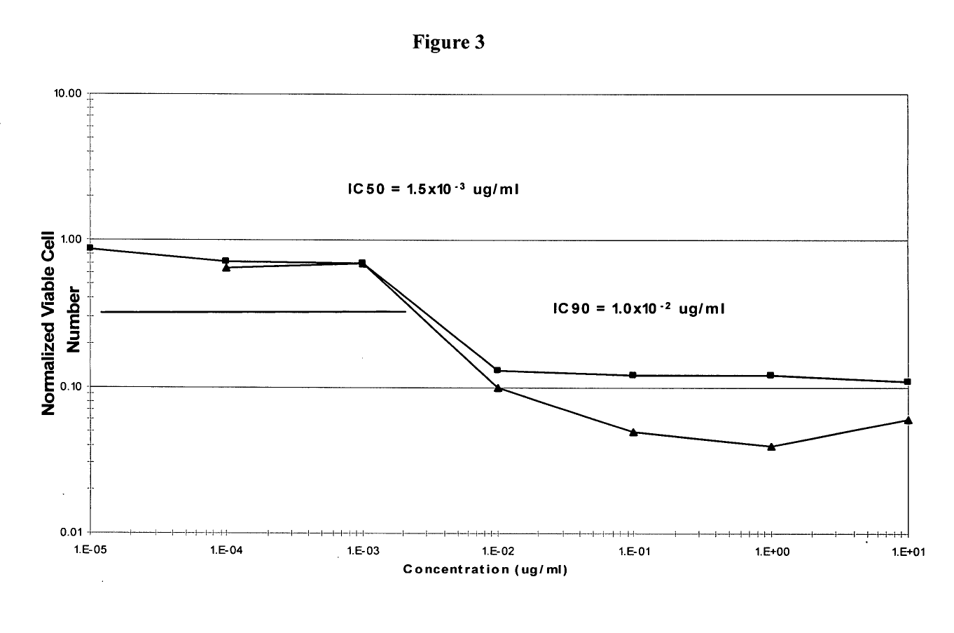

Human colorectal cancer HCT-116 cells were plated at 5×10⁴ cells in T25 tissue culture flasks (Falcon Plastics, New Jersey) with 5 mL media RPMI 1640 (Cellgro, Virginia), which was supplemented with 15% BCS (Hyclone, Utah), 5% Pen. Strep. and 5% Glutamine (Cellgro). After three days (cells in logarithmic growth phase; 5×10⁵ cells/flask), 15-O-(3′-hydroxy-3′-isopropyl-4′-methylpentanoyl)bruceolide, 15-(3-isopropyl-4-methyl-2-pentenoyl)-bruceolide, and, for comparative purposes, bruceantin, peninsularinone, or glaucarubolone was added to each flask to achieve concentrations ranging from 10¹ to 10⁻⁴ ug/mL. At day 3, the flasks were washed, trypsinized, and spun down. The resulting cells were counted for both viable and dead cells using 0.08% trypan blue (Gibco, Maryland). Viable cell number as a function of compound concentration was plotted and the IC₅₀ and IC₉₀ values were determined by interpolation. The IC₅₀ and IC₉₀ are the concentrations required to kill 50% and 90%, respectively, of the HCT-116 cells. The results of this analysis are set forth in Table 6. The cytotoxicity of 15-O-(3′-hydroxy-3′-isopropyl-4′-methylpentanoyl)bruceolide as a function of concentration is illustrated in FIG. 2. The cytotoxicity of 15-(3-isopropyl-4-methyl-2-pentenoyl)-bruceolide as a function of concentration is illustrated in FIG. 3.

TABLE 6

| | IC₅₀ | IC₉₀ | |
|---|---|---|---|
| Compound | (μg/mL) | (μg/mL) | IC₉₀/IC₅₀ |
| 15-O-(3′-hydroxy-3′-isopropyl-4′- | 1.9 × 10⁻³ | 6.7 × 10⁻² | 35 |
| methylpentanoyl)bruceolide | | | |
| (inventive) | | | |
| 15-O-(3′-isopropyl-4′-methyl-2′- | 1.5 × 10⁻³ | 1.0 × 10⁻² | 7 |
| pentenoyl)-bruceolide | | | |
| (inventive) | | | |
| Bruceantin | | | |
| (comparative) | 1.5 × 10⁻³ | 1.0 × 10¹ | 6,700 |
| Peninsularinone | 1.2 × 10⁻³ | 1.5 × 10⁻² | 13 |
| (comparative) | | | |
| Glaucarubolone | 4.0 × 10⁻² | 2.3 × 10⁰ | 58 |
| (comparative) | | | |

This example demonstrates that the inventive compounds induce potent cytoxicity in human colorectal cancer cells in vitro.

### Example 9

This example demonstrates a method of inhibiting clonogenic survival of tumor cells in vitro using the inventive compounds.

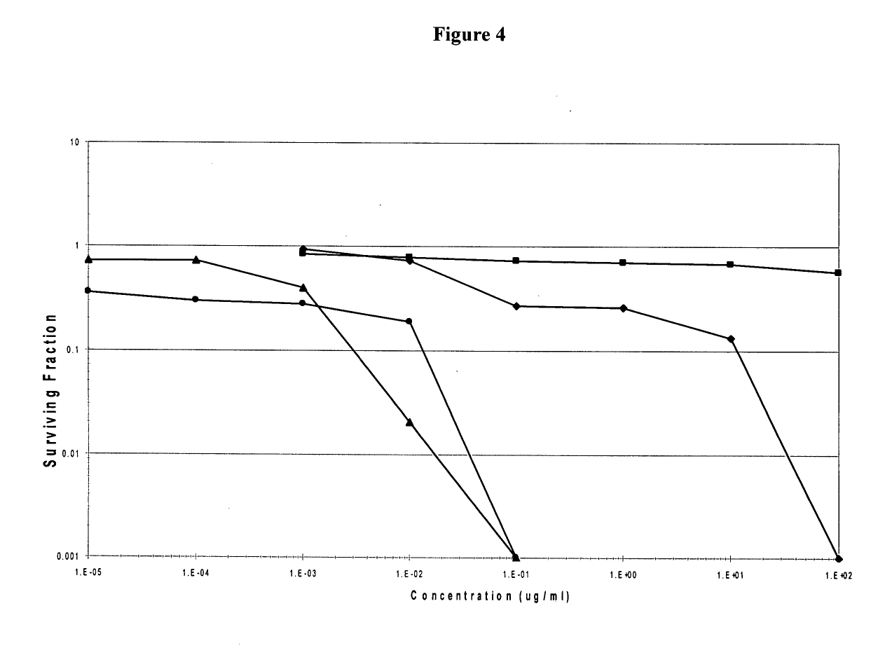

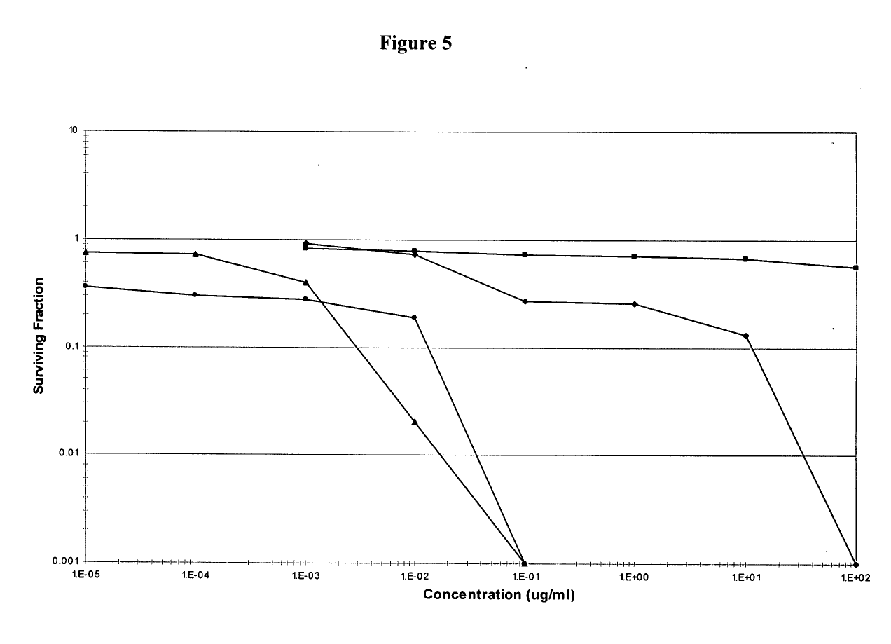

A clonogenic assay was performed using HCT-116 cells. Specifically, HCT-116 cells were seeded at 200 to 20,000 cells in 60 mm dishes. Samples of 15-O-(3′-hydroxy-3′-isopropyl-4′-methylpentanoyl)bruceolide, 15-(3-isopropyl-4-methyl-2-pentenoyl)-bruceolide, and, for comparative purposes, bruceantin, peninsularinone, and glaucarubolone were added to the culture medium (RPMI+10% FBS) to achieve a final concentration of 10 μg/mL and 10-fold dilutions thereof. At either 2 hours or 24 hours following drug administration, the drug-containing media was removed, and fresh media without drug was added. In addition, one group of cells was subjected to continuous drug exposure for the entire incubation period. The dishes were incubated for 7 days (at 37° C. and 5% CO₂), media was removed, and the colonies were stained with methylene blue. Colonies containing 50 cells or more were counted. The results were normalized to an untreated control. Plating efficiency for the untreated cells was about 90%. Repeat experiments also were carried out to define the cell survival range between 1 (100%) and 10⁻³ (0.1%) survival. A survival rate of 10⁻¹ was chosen as the minimal level that would be required in vivo to achieve therapeutic efficacy against HCT-116 cells in tumor-bearing mice. The results of this experiment are set forth in Table 7. The clonogenic dose response to 15-O-(3′-hydroxy-3′-isopropyl-4′-methylpentanoyl)bruceolide is illustrated in FIG. 4. The clonogenic dose response to 15-(3-isopropyl-4-methyl-2-pentenoyl)-bruceolide is illustrated in FIG. 5.

TABLE 7
Concentration Required for 10⁻¹ Survival Fraction (μg/mL)

| | 2 Hour | 24 Hour | Continuous |
|---|---|---|---|
| Compound | Exposure | Exposure | Exposure |
| 15-O-(3′-hydroxy-3′- | >10 | 1 × 10⁰ | 7 × 10⁻³ |
| isopropyl-4′- | | | |
| methylpentanoyl)bruceolide | | | |
| (inventive) | | | |
| 15-(3-isopropyl-4-methyl- | >10 | 1.5 × 10⁰ | 1.5 × 10⁻³ |
| 2-pentenoyl)-bruceolide | | | |
| (inventive) | | | |
| Glaucarubolone | >10 | 1 × 10⁰ | 2 × 10⁻² |
| (comparative) | | | |
| Peninsularinone | >10 | 1 × 10⁻² | 1 × 10⁻³ |
| (comparative) | | | |
| Bruceantin | >10 | >10 | 2 × 10⁻³ |
| (comparative) | | | |

This example demonstrates that continuous exposure of tumor cells to the inventive compounds is effective in killing tumor cells.

### Example 10

This example demonstrates the determination of the maximum tolerated dose of the inventive compounds in mice.

Formulations containing 15-O-(3′-hydroxy-3′-isopropyl-4′-methylpentanoyl)bruceolide, 15-(3-isopropyl-4-methyl-2-pentenoyl)-bruceolide, and, for comparative purposes, bruceantin, or peninsularinone were prepared by solubilizing each compound in up to 100 mg/mL of ethanol. These solutions were diluted 1:1 with a stabilizer (Cremophor/propylene glycol). The solutions were then diluted in saline (1:10 minimum).

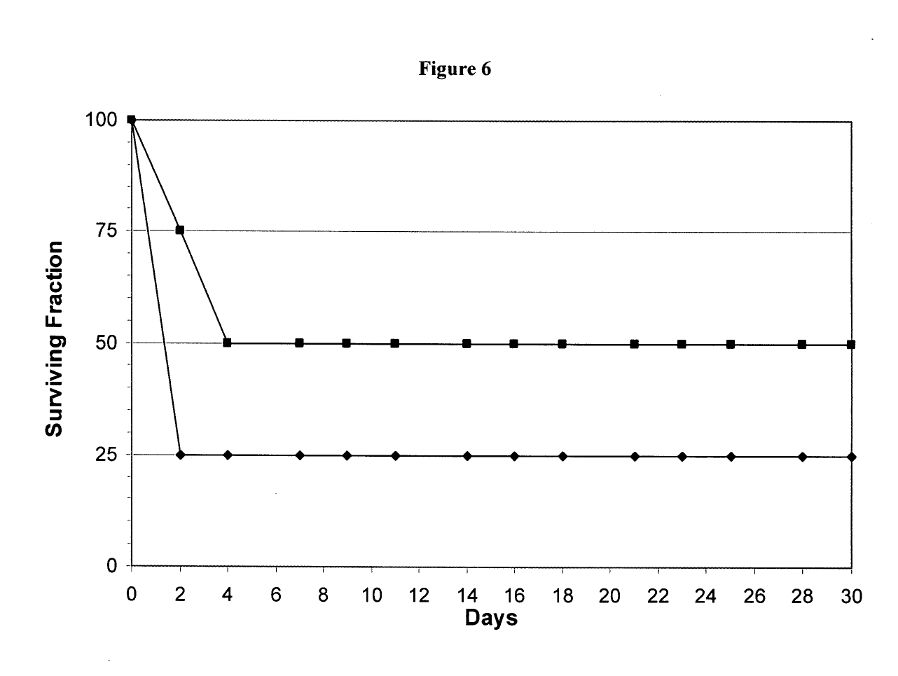

The maximum tolerated dose (MTD) was determined by a two-stage experiment. In the first stage, between 1 and 10 mg/mL of the formulation was prepared as described above. Next, 0.25 mL of the formulation was injected intravenously into one 20 gram mouse (0.75 mg/mouse). If the animal died within 30 days of injection, a series of subsequent 2-fold dilutions were administered to individual mice until an MTD was defined. If the mouse survived the initial dose then formulations containing higher drug dosages were prepared. The second stage of the study involved increasing the number of mice per dose to four, and retesting four dilutions that bracket the MTD from the individual mouse study. The four dilutions were the MTD, a dosage twice the MTD, a dosage ½ the MTD, and a dosage ¼ the MTD. The results of this experiment are shown in Table 8 and FIG. 6. In FIG. 6, at a dose of either 1.5 or 0.75 mg/mouse 15-O-(3′-hydroxy-3′-isopropyl-4′-methylpentanoyl)bruceolide, 100% of the mice survived the duration of the experiment. Similarly, at a dose of 0.1, 0.075, 0.05, or 0.025 mg/mouse 15-O-(3-isopropyl-4-methyl-2-pentenoyl)-bruceolide, 100% of the mice survived the duration of the experiment.

TABLE 8

| | Dose - 1 Mouse | | Dose - 4 Mice | |
|---|---|---|---|---|
| Compound | mg/mouse | mg/kg | mg/mouse | mg/kg |
| Peninsularinone | 0.01 | 0.45 | 0.005 | 0.25 |
| (comparative) | | | | |
| Bruceantin | 0.1 | 5.4 | — | — |
| (comparative) | | | | |
| 15-O-(3′-hydroxy-3′- | 3.2 | 109 | 1.5 | 60 |
| isopropyl-4′- | | | | |
| methylpentanoyl)bruceolide | | | | |
| (inventive) | | | | |
| 15-(3-isopropyl-4-methyl- | 0.05 | 2.9 | 0.25 | 4 |
| 2-pentenoyl)-bruceolide | | | | |
| (inventive) | | | | |

This example demonstrates that 15-O-(3′-hydroxy-3′-isopropyl-4′-methylpentanoyl)bruceolide (MTD 60-65 mg/kg/day) was the least toxic of the tested compounds.

### Example 11

This example demonstrates the pharmacokinetic properties of the inventive compounds.

Samples from HCT-116 tumor-bearing SCID mice were analyzed via high performance liquid chromatography (HPLC) following administration of inventive compositions. Specifically, 15-O-(3′-hydroxy-3′-isopropyl-4′-methylpentanoyl)bruceolide and 15-(3-isopropyl-4-methyl-2-pentenoyl)-bruceolide were administered intravenously (2.25 mg/mouse) into five tumor-bearing mice and five control mice. At different time points following administration of the inventive compounds (up to 24 hours), individual mice were killed and both blood and tumor samples were removed for HPLC analysis.

In particular, a Waters 2690 Separation Module (Waters Corp., Milford, Mass.) connected to a Waters 2487 Dual wavelength absorbance detector controlled by Millennium32 v3.20 was used for separation, detection, data acquisition, integration and quantitation. An external standard calibration method was used to quantify the inventive compounds in each sample. A Symmetry Shield RP18 5 μm (4.9×150 mm) analytical column (Waters Corp., Milford, Mass.) was used in the separation of each inventive compound. The column temperature was maintained at 30° C. 25 μl of each sample was injected per analytical run.

The mobile phase consisted of 99% water/1% acetonitrile as the initial gradient composition for the inventive composition. A gradient analysis from deionized water to acetonitrile at a flow rate of 1 mL/min for each tested compound is set forth in Tables 9 and 10. The detection wavelength for the inventive composition was 278 nm, and the retention time was 7.8 minutes.

TABLE 9
15-O-(3′-hydroxy-3′-isopropyl-4′-
methylpentanoyl)bruceolide

| Time | Flow | % A | % B | % C | % D | Curve |
|---|---|---|---|---|---|---|
| | 1.00 | 0.0 | 99.0 | 1.0 | 0.0 | |
| 7.80 | 1.00 | 0.0 | 21.0 | 79.0 | 0.0 | 6 |
| 9.00 | 1.00 | 0.0 | 21.0 | 79.0 | 0.0 | 6 |
| 15.00 | 1.00 | 0.0 | 99.0 | 1.0 | 0.0 | 6 |

TABLE 10
15-(3-isopropyl-4-methyl-2-pentenoyl)-bruceolide

| Time | Flow | % A | % B | % C | % D | Curve |
|---|---|---|---|---|---|---|
| | 1.00 | 0.0 | 100.0 | 0.0 | 0.0 | |
| 10.00 | 1.00 | 0.0 | 0.0 | 100.0 | 0.0 | 6 |
| 15.00 | 1.00 | 0.0 | 100.0 | 0.0 | 0.0 | 6 |

For the in vivo pharmacokinetic trial, 15 mice were inoculated bilaterally and subcutaneously in the inguinal area with 10⁷ tumor cells (by a 16-gauge needle), such as HCT-116 cells or trocar-implanted Colon 38 cells. Each of the compounds 15-O-(3′-hydroxy-3′-isopropyl-4′-methylpentanoyl)bruceolide and 15-(3-isopropyl-4-methyl-2-pentenoyl)-bruceolide was administered intravenously at the MTD in 0.25 mL via the tail vein. Each compound was administered to 10 animals when the tumors reached a measurable size (i.e., 80 to 300 mm³) at a dose determined from the MTD study. The remaining five animals served as time-point controls. At prescribed times thereafter, one animal per time point was sacrificed, and blood and bilateral tumors were harvested for analysis of drug concentrations. Specifically, mice were sacrificed for pharmacological analysis at 1, 5, 15, 30, 60, 120, 240, 360 minutes and 12 and 24 hours following administration of the inventive compounds.

At one hour post administration, the concentration of 15-O-(3′-hydroxy-3′-isopropyl-4′-methylpentanoyl)bruceolide in both plasma and tumor samples was approximately 7 μg/mL. By two hours post administration, concentration of this compound in the plasma samples had decreased to 1.5 μg/mL; however, the concentration of the compound in the tumor samples was approximately 4 μg/mL. By six hours post administration, the concentration of 15-O-(3′-hydroxy-3′-isopropyl-4′-methylpentanoyl)bruceolide in the plasma samples had decreased to 100 ng/mL, while the concentration in the tumor sample was 800 ng/mL. The mouse designated for analysis at 24 hours post administration did not survive, as the dose of 15-O-(3′-hydroxy-3′-isopropyl-4′-methylpentanoyl)bruceolide administered was above the MTD.

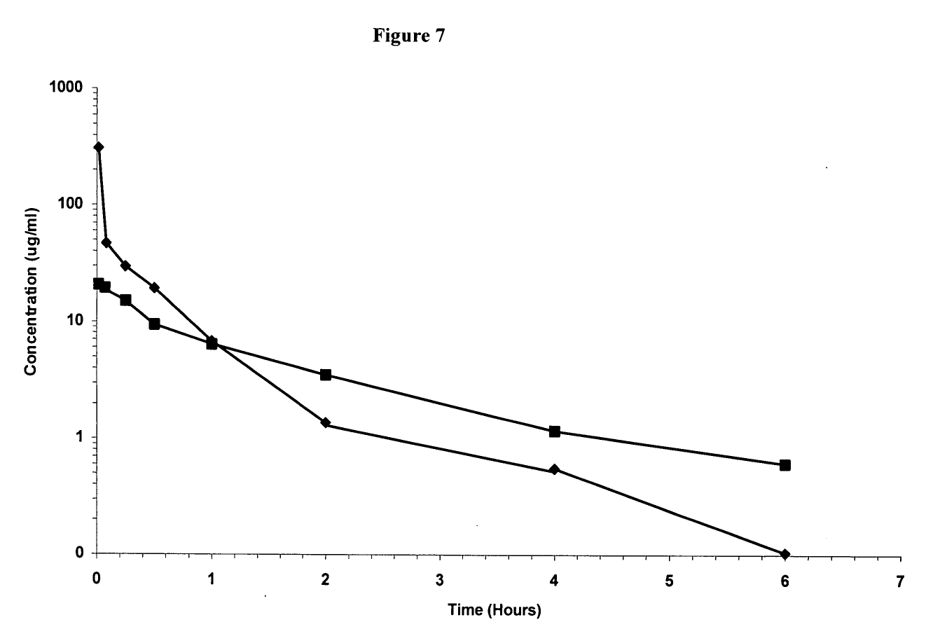

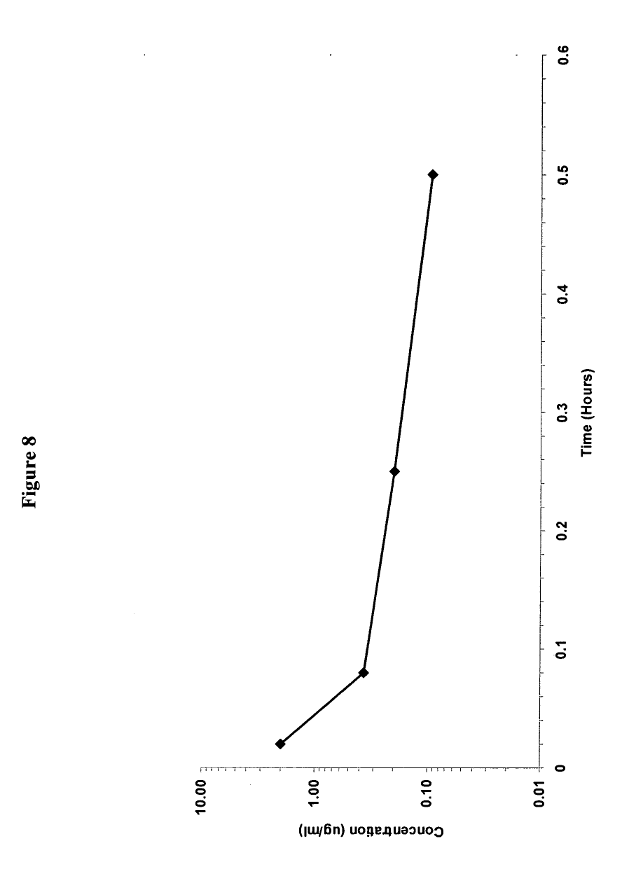

For 15-(3-isopropyl-4-methyl-2-pentenoyl)-bruceolide, plasma concentrations could only be assessed for the 0.5 hour time point (100 ng/mL), and were below the sensitivity of the assay at the 1 hour time point (i.e., 50 ng/mL). The concentration of this compound in tumor samples also was below the sensitivity level of the assay for the entire 24 hour sampling period. The concentration of 15-O-(3′-hydroxy-3′-isopropyl-4′-methylpentanoyl)bruceolide over time in vivo is illustrated in FIG. 7. The concentration of 15-(3-isopropyl-4-methyl-2-pentenoyl)-bruceolide over time in vivo is illustrated in FIG. 8.

The results of this example demonstrate that repeated administration of 15-O-(3′-hydroxy-3′-isopropyl-4′-methylpentanoyl)bruceolide at the MTD can produce sufficient concentrations of the compound in the tumor and plasma to yield a therapeutic effect.

### Example 12

This example demonstrates a method of inhibiting tumor growth in vivo using an inventive compound.

SCID mice weighing more than 17 grams were supplied food and water ad libidum. The animals were pooled, implanted subcutaneously with tumor cells, and pooled again before distribution to treatment and control groups. Treatment was started when the tumors were palpable (i.e., 10 days after inoculation). Mice received one of four doses (i.e., 1.6, 1.2, 0.8, and 0.4 mg/mouse) of the inventive compound. Each dose was administered intravenously via the tail vein once per day for 5 days. Mice were killed when their tumors reached 1500 mm³.

Tumor weights were estimated from two-dimensional caliper measurements performed twice a week according to the following formula:
Tumor Weight (mg)=(a×b²)/2
in which a and b are the tumor length and width, respectively, in millimeters.

Therapeutic efficacy was measured as the % T/C value (T=treated, C=control), which was determined each time a tumor measurement was made. The median tumor weight of the treatment and control groups was determined. A % T/C value equal to or less than 50% indicates antitumor activity. A % T/C value of <10% indicates highly significant antitumor activity, and is the level used by the National Cancer Institute (NCI) to justify a clinical trial if toxicity, formulation and other requirements are met. A weight loss of greater than 20% (mean of group) or greater than 20% drug deaths indicates an excessively toxic dose.

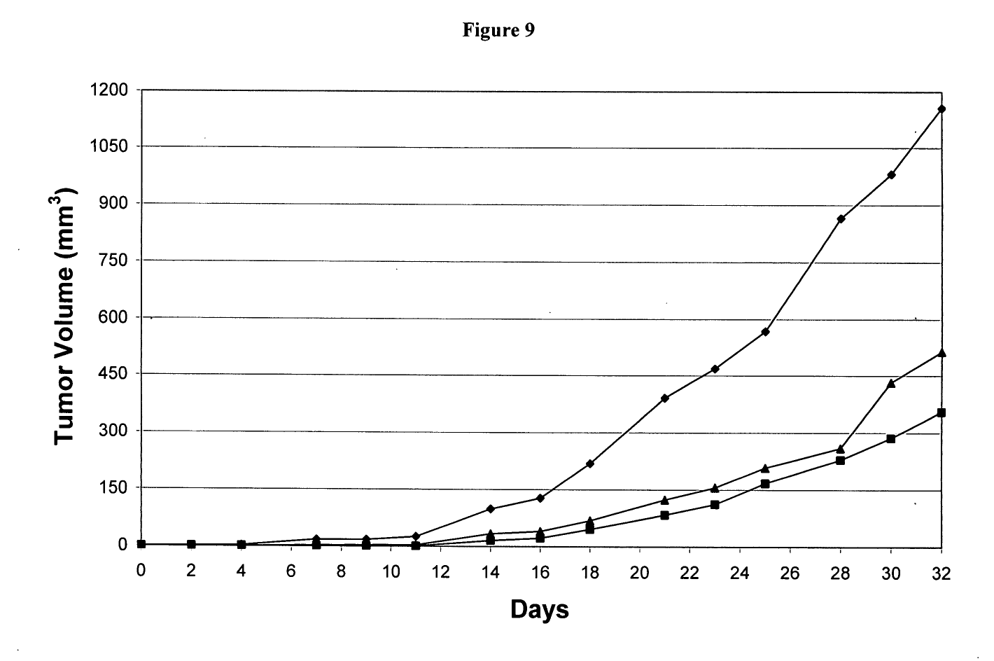

The highest two doses as well as one mouse in the third highest dose died after the five days of treatment. The remaining nine mice in the lower two groups survived for 30 days, producing a significant therapeutic effect with a % T/C=16% for the higher dose group and 31% for the lower dose group. The results of this analysis are set forth in FIG. 9.

This example demonstrates that the inventive compound is effective in inhibiting tumor growth in vivo.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A compound of the formula (I) 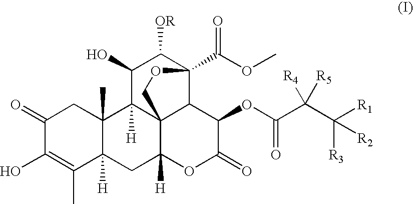
wherein R is selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl,
wherein R₁, R₂, and R₃ are independently selected from the group consisting of hydrogen, halo, alkyl, alkenyl, alkynyl, cycloalkyl, cycloalkenyl, aryl, arylalkyl, carboxyl, carbonyl, hydroxyalkyl, alkoxyalkyl, —OR₆, and —NR₉R₁₀, or wherein R₁ and R₂ taken together form C₃-C₁₀ alkylene, C₃-C₁₀ alkenylene, C₃-C₁₀ heterocycloalkyl, or C₃-C₁₀ heteroaryl,
wherein R₄ and R₅ are independently selected from the group consisting of hydrogen, alkyl, alkenyl, alkynyl, hydroxyalkyl, alkoxyalkyl, cycloalkyl, cycloalkenyl, aryl, and arylalkyl, or wherein R₄ and R₅ taken together form C₃-C₁₀ alkylene or alkenylene, or wherein R₄ and R₅ taken together form (C═O),
wherein R₆ is selected from the group consisting of hydrogen, alkyl, alkenyl, alkynyl, hydroxyalkyl, alkoxyalkyl, cycloalkyl, cycloalkenyl, aryl, arylalkyl, and —C(O)R₇,
wherein R₇ is selected from the group consisting of hydrogen, alkyl, alkenyl, alkynyl, hydroxyalkyl, alkoxyalkyl, cycloalkyl, cycloalkenyl, aryl, arylalkyl, —OR₈, and —NR₉R₁₀,
wherein R₈ is selected from the group consisting of alkyl, alkenyl, alkynyl, cycloalkyl, cycloalkenyl, aryl, and arylalkyl,
wherein R₉ and R₁₀ are independently selected from the group consisting of hydrogen, alkyl, alkenyl, alkynyl, hydroxyalkyl, alkoxyalkyl, cycloalkyl, cycloalkenyl, aryl, aralkyl, alkylcarbonyl, and arylcarbonyl, or R₉ and R₁₀ can be taken together to form a 5-7 membered heterocyclic ring with the nitrogen to which they are bonded,
with the provisos (i) R₁, R₂, R₃, R₄ and R₅ are not all hydrogen; (ii) when R₄ and R₅ are hydrogen and R₃ is hydroxyl, then R₁ and R₂ are not both methyl; (iii) if R₄ and R₅ are hydrogen and R₁ is methyl, then R₂ is not hydrogen and R₃ is not trifluoromethyl, (iv) when R₄ and R₅ are hydrogen and R₁ is isopropyl, then R₂ is not hydrogen and R₃ is not methyl, and (v) when R₃, R₄ and R₅ are hydrogen, then R₁ and R₂ are not both methyl.

2. The compound of claim 1, wherein R₁ and R₂ are independently selected from the group consisting of hydrogen, alkyl, alkenyl, alkynyl, cycloalkyl, cycloalkenyl, aryl, arylalkyl, carboxyl, carbonyl, hydroxyalkyl, and alkoxyalkyl, and
wherein R₃ is —OR_{6.}

3. The compound of claim 2, wherein R₁ and R₂ are ethyl, R₃ is hydroxyl, and R₄ and R₅ are hydrogen.

4. The compound of claim 2, wherein R₁ and R₂ are isopropyl, R₃ is hydroxyl, and R₄ and R₅ are hydrogen.

5. The compound of claim 1, wherein R₁, R₂, and R₃ are methyl and R₄ and R₅ are hydrogen.

6. The compound of claim 1, wherein R₁ is hydroxyl, R₂ is carboxy or an alkyl ester derivative thereof, and R₃ is hydrogen or methyl.

7. The compound of claim 2, wherein R₄ and R₅ taken together form a four-membered cycloalkane, R₁ and R₂ are both hydrogen, and R₃ is hydroxyl.

8. The compound of claim 1, wherein R₃ is —(CH₂)ₙOR₆, wherein n is an integer of 1-10.

9. The compound of claim 8, wherein R₁ and R₂ are ethyl, R₃ is hydroxymethyl, and R₄ and R₅ are hydrogen.

10. The compound of claim 8, wherein R₁ and R₂ are isopropyl, R₃ is hydroxymethyl, and R₄ and R₅ are hydrogen.

11. The compound of claim 8, wherein R₁ and R₂ are methyl, R₃ are hydroxymethyl, and R₄ and R₅ are hydrogen.

12. A compound of the formula (II) 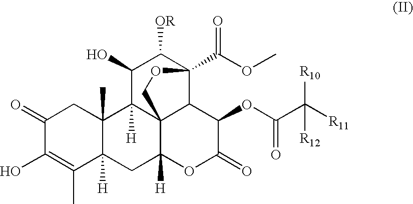
wherein R is selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl,
wherein R₁₀ , R₁₁, and R₁₂ are independently selected from the group consisting of hydrogen, halo, alkyl, alkenyl, alkynyl, cycloalkyl, cycloalkenyl, aryl, arylalkyl, carboxyl, carbonyl, hydroxyalkyl, alkoxyalkyl, and —OR₁₃, or wherein R₁₀ and R₁₁ taken together form C₃-C₁₀ alkylene, C₃-C₁₀ alkenylene, or C₃-C₁₀ heterocycloalkyl,
wherein R₁₃ is selected from the group consisting of hydrogen, alkyl, alkenyl, alkynyl, hydroxyalkyl, alkoxyalkyl, cycloalkyl, cycloalkenyl, aryl, arylalkyl, and —C(O)R₁₄,
wherein R₁₄ is selected from the group consisting of alkyl, alkenyl, alkynyl, hydroxyalkyl, alkoxyalkyl, cycloalkyl, cycloalkenyl, aryl, arylalkyl, —OR₁₅, and —NR₁₆R₁₇,
wherein R₁₅ is selected from the group consisting of alkyl, alkenyl, alkynyl, cycloalkyl, cycloalkenyl, aryl, and arylalkyl,
wherein R₁₆ and R₁₇ are independently selected from the group consisting of hydrogen, alkyl, alkenyl, alkynyl, hydroxyalkyl, alkoxyalkyl, cycloalkyl, cycloalkenyl, aryl, and arylalkyl, or R₁₆ and R₁₇ can be taken together to form a 5-7 membered heterocyclic ring with the nitrogen to which they are bonded,
with the proviso that R, R₁₀, R₁₁, and R₁₂ are not all hydrogen.

13. The compound of claim 12, wherein R₁₀ and R₁₁ are independently selected from the group consisting of alkyl, alkenyl, alkynyl, cycloalkyl, cycloalkenyl, aryl, arylalkyl, carboxyl, carbonyl, hydroxyalkyl, and alkoxyalkyl,
wherein R₁₂ is —OR₁₃.

14. The compound of claim 12, wherein R₁₀ , R₁₁, and R₁₂ are methyl.

15. The compound of claim 13, wherein R₁₀ and R₁₁ are methyl and R₁₂ is hydroxyl.

16. The compound of claim 13, wherein R₁₀ and R₁₁ are isopropyl and R₁₂ is hydroxyl.

17. The compound of claim 12, wherein R₁₂ is —(CH₂)ₙOR₁₃, wherein n is an integer of 1-10.

18. The compound of claim 17, wherein R₁₀ and R₁₁ are methyl and R₁₂ is hydroxymethyl.

19. The compound of claim 17, wherein R₁₀ and R₁₁ are isopropyl and R₁₂ is hydroxymethyl.

20. A compound of the formula (III) 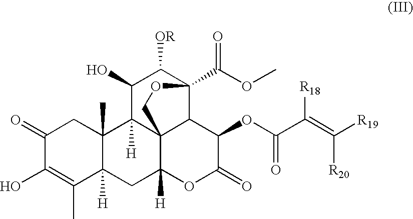
wherein R is selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl,
wherein R₁₈, R₁₉, and R₂₀ are independently selected from the group consisting of hydrogen, halo, alkyl, alkenyl, alkynyl, cycloalkyl, cycloalkenyl, aryl, arylalkyl, carboxyl, carbonyl, hydroxyalkyl, alkoxyalkyl, and —OR₂₁, or wherein R₁₉ and R₂₀ taken together form C₃-C₁₀ alkylene, C₃-C₁₀ alkenylene, or C₃-C₁₀ heterocycloalkyl,
wherein R₂₁ is selected from the group consisting of hydrogen, alkyl, alkenyl, alkynyl, hydroxyalkyl, alkoxyalkyl, cycloalkyl, cycloalkenyl, aryl, arylalkyl, and —C(O)R₂₂,
wherein R₂₂ is selected from the group consisting of alkyl, alkenyl, alkynyl, hydroxyalkyl, alkoxyalkyl, cycloalkyl, cycloalkenyl, aryl, arylalkyl, —OR₂₃, and —NR₂₄R₂₅,
wherein R₂₃ is selected from the group consisting of alkyl, alkenyl, alkynyl, cycloalkyl, cycloalkenyl, aryl, and arylalkyl,
wherein R₂₄ and R₂₅ are independently selected from the group consisting of hydrogen, alkyl, alkenyl, alkynyl, hydroxyalkyl, alkoxyalkyl, cycloalkyl, cycloalkenyl, aryl, and arylalkyl, or R₂₄ and R₂₅ can be taken together to form a 5-7 membered heterocyclic ring with the nitrogen to which they are bonded
with the proviso when R and R₁₈ are hydrogen and R₁₉ is methyl, then R₂₀ is not methyl, isopropyl, —C(CH₃)₂OH, or —C(CH₃)₂OAc.

21. The compound of claim 20, wherein R₁₈ is hydrogen and R₁₉ and R₂₀ are isopropyl.

22. A compound of the formula (IV) 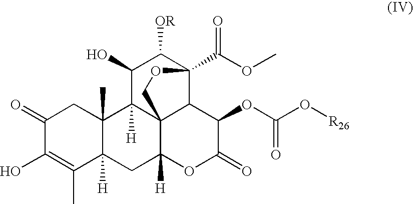
wherein R and R₂₆ are independently selected from the group consisting of hydrogen, alkyl, alkenyl, alkynyl, cycloalkyl, cycloalkenyl, C₃-C₁₀ heterocycloalkyl, aryl, C₃-C₁₀ heteroaryl, arylalkyl, carboxyl, hydroxyalkyl, and alkoxyalkyl.

23. A composition comprising a pharmaceutically suitable carrier and at least one compound of claim 1.

24. A method of treating cancer in a mammal, which method comprises administering to the mammal a dose of a composition comprising a pharmaceutically acceptable carrier and an effective amount of the composition of claim 23 to a mammal, whereupon the cancer is treated.

25. The method of claim 24, wherein the cancer is a solid tumor.

26. The method of claim 25, wherein the solid tumor is a colon tumor, a lung tumor, or a breast tumor.

27. The method of claim 24, wherein the cancer is a hematologic malignancy.

28. The method of claim 27, wherein the hematologic malignancy is selected from the group consisting of acute myeloid leukemia, acute lymphoblastic leukemia, blast-phase chronic myeloid leukemia, Burkitt's leukemia, Burkitt-like leukemia, and high-risk myelodysplastic syndrome.

29. A composition comprising a pharmaceutically suitable carrier and at least one compound of claim 12.

30. A method of treating cancer in a mammal, which method comprises administering to the mammal a dose of a composition comprising a pharmaceutically acceptable carrier and an effective amount of the composition of claim 29 to a mammal, whereupon the cancer is treated.

31. The method of claim 30, wherein the cancer is a solid tumor.

32. The method of claim 31, wherein the solid tumor is a colon tumor, a lung tumor, or a breast tumor.

33. The method of claim 30, wherein the cancer is a hematologic malignancy.

34. The method of claim 33, wherein the hematologic malignancy is selected from the group consisting of acute myeloid leukemia, acute lymphoblastic leukemia, blast-phase chronic myeloid leukemia, Burkitt's leukemia, Burkitt-like leukemia, and high-risk myelodysplastic syndrome.

35. A composition comprising a pharmaceutically suitable carrier and at least one compound of claim 20.

36. A method of treating cancer in a mammal, which method comprises administering to the mammal a dose of a composition comprising a pharmaceutically acceptable carrier and an effective amount of the composition of claim 35 to a mammal, whereupon the cancer is treated.

37. The method of claim 36, wherein the cancer is a solid tumor.

38. The method of claim 37, wherein the solid tumor is a colon tumor, a lung tumor, or a breast tumor.

39. The method of claim 36, wherein the cancer is a hematologic malignancy.

40. The method of claim 39, wherein the hematologic malignancy is selected from the group consisting of acute myeloid leukemia, acute lymphoblastic leukemia, blast-phase chronic myeloid leukemia, Burkitt's leukemia, Burkitt-like leukemia, and high-risk myelodysplastic syndrome.

41. A composition comprising a pharmaceutically suitable carrier and at least one compound of claim 22.

42. A method of treating cancer in a mammal, which method comprises administering to the mammal a dose of a composition comprising a pharmaceutically acceptable carrier and an effective amount of the composition of claim 41 to a mammal, whereupon the cancer is treated.

43. The method of claim 42, wherein the cancer is a solid tumor.

44. The method of claim 43, wherein the solid tumor is a colon tumor, a lung tumor, or a breast tumor.

45. The method of claim 42, wherein the cancer is a hematologic malignancy.

46. The method of claim 45, wherein the hematologic malignancy is selected from the group consisting of acute myeloid leukemia, acute lymphoblastic leukemia, blast-phase chronic myeloid leukemia, Burkitt's leukemia, Burkitt-like leukemia, and high-risk myelodysplastic syndrome.
